(19) United States
(12) Patent Application Publication
(10) Pub. No.: US 2007/0284250 A1
(43) Pub. Date: Dec. 13, 2007

(54) Multifunctional Electrophoresis Cassette

(75) Inventors: Gary P. Magnant, Topsfield, MA (US); Michael Finney, San Francisco, CA (US); Todd J. Barbera, Marblehead, MA (US); William Bowers, Ipswich, MA (US); Diane Kozwich, Nottingham, NH (US); Samuel Seymour, Marblehead, MA (US); Robert J. Nelson, Sioux Falls, SD (US); T. Christian Boles, Bedford, MA (US)

Correspondence Address: BROMBERG & SUNSTEIN LLP, 125 SUMMER STREET, BOSTON, MA, 02110-1618, US

(73) Assignee: SAGE SCIENCE, INC., Beverly, MA (US)

(21) Appl. No.: 11/685,634

(22) Filed: Mar. 13, 2007

Related U.S. Application Data
(60) Provisional application No. 60/781,874, filed on Mar. 13, 2006.

(51) Int. Cl. C07K 1/26; C07K 1/28; C25B 9/08

(52) U.S. Cl. 204/459; 204/456; 204/461; 204/464; 204/606; 204/610; 204/614; 73/864.91

(57) ABSTRACT

Devices and methods are provided for more efficiently performing electrophoresis and electroblotting. A sandwich structure includes an electrophoresis gel affixed to a blotting membrane. A gel casting and/or running frame is used to hold the sandwich. A method and composition that allows separation of the gel and membrane after performing a combined electrophoresis and electroblotting operation so as to allow further operations to be performed individually upon the membrane, gel or both. A uniform electrophoretic field may be created by surrounding the sandwich structure using an insulating fluid; the insulating fluid is then swapped for a conducting fluid to allow application of an electroblotting field. An apparatus automatically manages fluid exchange and actuation of electrophoresis and electroblotting electrodes. A plurality of parallel cavities may be used to hold multiple gels or gel membrane sandwiches.

### CROSS REFERENCE TO RELATED APPLICATIONS

This application claims priority from the following U.S. Provisional Patent Application, Ser. No. 60/781,874 for “Multifunctional Electrophoresis Cassettes and Instruments” filed Mar. 13, 2006 (Attorney Docket No. 3094/101);

### TECHNICAL FIELD

The present invention relates to devices and methods for performing parallel or sequential electrophoresis and electroblotting operations for purposes including molecular biological applications.

### BACKGROUND

It is a common practice in biological experimentation to separate macromolecules such as proteins and nucleic acids, e.g., DNA or RNA, for analytical and preparative purposes using electrophoresis. Electrophoresis separates biomolecules by charge and/or size via mobility through a separating matrix in the presence of an electric field. Gel separating matrices are typically prepared from agarose for nucleic acid separation and polyacrylamide for protein separation. In capillary electrophoresis, the matrices may be gels or solutions (e.g., linear polyacrylamide solution).

Gel separating matrices are typically made by pouring a liquid phase material into a mold formed by glass plates or separating matrix casting molds. In slab gel electrophoresis, for example, finger shaped outcroppings in plastic material form “combs” that are embedded in the top of the separating matrix. Sample loading wells are formed when the combs are removed from the solidified separating matrix. Loading these wells is typically a time consuming and technically challenging task. Dense solutions such as glycerol or polyethylene glycol are often added to samples prior to electrophoresis to prevent samples from mixing with electrode buffers and floating out of the wells.

Samples, generally in an aqueous buffer, are applied to the separating matrix and electrodes in electrical contact with the separation matrix are used to apply an electric field. The field induces charged materials, such as nucleic acids and proteins, to migrate toward respective anode or cathode positions. Electrophoresis is usually completed in about 30 minutes to several hours.

The migration distances for the separated molecular species depend on their relative mobility through the separating matrix. Mobility of each species depends on hydrodynamic size and molecular charge. Proteins are often electrophoresed under conditions where each protein is complexed with a detergent or other material that imparts a negative charge to proteins in the sample. The detergent causes most or all of the proteins to migrate in the same direction (toward the electrophoresis anode). Samples may be stained prior to, during, or after a separation run to visualize the nucleic acids or proteins within the gel. The location of the various components in the gel is determined using ultraviolet light absorbance, autoradiography, fluorescence, chemiluminescence, or any other well known means of detection. To determine the molecular weight and relative concentration of unknown nucleic acids or proteins, the band positions and intensities are typically compared to known molecular standards.

Blotting is a process used to transfer macromolecules from an electrophoresis matrix to a membrane for further analysis, such as Southern, Northern, or Western blotting. Traditionally the separating matrix containing the electrophoresed biological material is removed from the electrophoresis apparatus and placed in a blotting sandwich. The blotting sandwich generally consists of buffer saturated sponges and paper pads; a gel containing the separated biologicals; a suitable transfer membrane that is in intimate contact with the separating matrix; and another layer of buffer saturated paper pads and sponges. In electroblotting, electrotransfer electrodes and buffer may provide an electric field to move the biologicals out of the separating matrix and into the membrane.

Electrophoresis and electroblotting are usually performed in separate apparatus because the electrode plane orientation for electrophoresis should be perpendicular to that of electrotransfer electrode plane orientation. During electrophoresis, the electrode placements are at the end containing the sample and the end opposite the sample. Parallel glass plates or plastic cassettes containing the separating matrix act as insulators and confine the current generated by the electrodes to the plane of the gel. A membrane is the aligned with the gel and electrotransfer electrodes are placed in an orientation that is perpendicular to the electrode orientation used for the electrophoretic separation. Since the glass and plastic used to contain the separating matrix are insulators, the glass plates or plastic cassette must be disassembled for transfer to take place.

U.S. Pat. No. 4,889,606 to Dyson, the full disclosure of which is hereby incorporated herein by reference, teaches a device and method using a gel and membrane containing sandwich structures to accomplish a two-stage electrophoresis and electroblotting.

### SUMMARY OF THE INVENTION

A combined electrophoresis and blotting assembly has a frame with at least one window, a first membrane and a gel adjacent to the membrane. The membrane is attached to the frame so as to extend across the window.

In related embodiments, the first membrane may be a blotting membrane. Alternately, the blotting membrane may also be positioned between the first membrane and the gel. The frame may have a plurality of windows. The membrane may be attached to the frame via a polymeric material of the frame that is dissolved within the pores of the membrane adjacent to the frame. The membrane may be chemically tensioned across the window.

In another embodiment, a combined electrophoresis and blotting assembly has a first gel and a blotting membrane layered upon the gel. The blotting membrane is affixed to the gel by a second peelable gel. The first gel may be a polyacrylamide gel and the peelable gel may be an agarose gel.

In a related embodiment, a structure for sequential electrophoresis and electroblotting has a gel cast between two membranes and each membrane is solvent-welded and chemically tensioned to a frame. At least one of the membranes as may be coated with a release agent.

In a further embodiment, there is a method for performing electrophoresis and blotting that includes the steps of providing an electrophoresis gel with a blotting membrane adjacent to the gel. The membrane defines an electrophoresis plane. The gel and membrane are immersed in an electrically insulating liquid and a first electric field having at least a component oriented along the electrophoresis plane is applied. The field is of sufficiently high voltage to cause electrophoretic mobility of charged analyte molecules in the gel. The insulating liquid is replaced with an electrically conductive liquid. A second electric field is applied; the field has a component normal to the electrophoretic plane and is of sufficiently high voltage so as to cause migration of the analyte molecules to the membrane.

In related embodiments, the gel may be separated from the membrane after migration of the molecules to the membrane. The membrane may also include a release agent. The membrane may be solvent welded to a frame so as to extend across at least one window. The membrane may be chemically tensioned to the frame. The frame may have a plurality of windows.

In another embodiment, an electrophoresis and electroblotting instrument has a jig for holding an electrophoretic gel adjacent to a blotting membrane. A first electrode pair is oriented to apply an electrophoretic field within the gel. A second electrode pair is oriented to apply an electroblotting field across the gel and membrane. A fluidic line has a first reservoir for holding an insulating fluid, a second reservoir for holding a conducting electrolyte fluid, and conduits for transporting the insulating fluid and the conducting fluid to regions proximal to the gel and the membrane. An automatically actuable fluid delivery assembly is adapted to selectively introduce either the insulating or conducting fluid to the gel and membrane. The instrument has circuitry for sequentially actuating the introduction of insulating fluid, the first electrode pair, the introduction of conducting fluid, and the second electrode pair so as to first effectuate electrophoresis in the presence of the insulating fluid and then effectuate electroblotting in the presence of the conducting fluid. The instrument may also have a cooler to remove heat from any or all of the insulating fluid, the conduit, the fluid and the gel.

In another related embodiment, a system for parallel gel electrophoresis includes at least one cassette with a plurality of cavities. The cavities are adapted to hold a plurality of electrophoresis separation matrices and each cavity has a corresponding individual sample loading port. The sample loading ports are arranged with a microplate spacing.

In another related embodiment a system for parallel gel electrophoresis has a least one cassette with a plurality of cavities that hold a plurality of electrophoretic separation matrices. Each cavity has a corresponding individual sample loading port. The sample loading ports are arranged with microplate spacing

In yet another embodiment there is a method for sample analysis and processing that has the steps of: providing a least one cassette with a plurality of gel cavities that hold a plurality of gels, wherein each gel cavity has a corresponding individual sample loading port; forming a gel in each of the plurality of cavities; introducing a plurality of samples into a plurality of corresponding loading ports; and performing electrophoresis. At least one gel is bounded by a membrane.

In another embodiment, an expandable microplate-format frame has a plurality of receptacles arranged in a configuration selected from the group consisting of 8 rows of 12 receptacles, and 12 rows of 8 receptacles; and a means for increasing the distance between the rows of receptacles.

In yet another embodiment, there is a system for electrophoresis. The system has a frame with a plurality of elongate projections that extend substantially parallel to a given plane. The projections define at least one gel cavity that is filled by at least one corresponding gel. A membrane bounds the gel on at least one side and is in a plane substantially parallel to the given plane. The membrane is removably attachable to the gel.

In a related embodiment, there is a method of electrophoresis that includes: providing a frame having a strip of electrophoretic gels bounded by and attached to a membrane; using the gels to perform gel electrophoresis; and removing the membrane so as to remove the electrophoretic gels attached to the membrane from the frame.

In yet another embodiment, there is an electrophoresis system. The system includes at least one electrophoretic gel that has a first terminus and a second terminus bounding a continuous, non-linear gel path and a first upward-opening port for holding a liquid. The bottom of the first port is bounded by the first terminus of the gel to form a first well. The system has a second upward-opening port for holding a liquid. The bottom of the second port is bounded by the second terminus of the gel to form a second well. The nadir of the gel path is below either one of the first terminus or the second terminus.

In another embodiment, there is a system for two dimensional electrophoresis. The system includes an elongate immobilized pH gradient member and a complementary parallel electrophoresis cassette having a plurality of longitudinally arranged gel cavities for holding a plurality of separation matrices. At least one cavity has a corresponding individual sample loading port with walls. The plurality of gel cavities and sample loading ports are in a lateral arrangement. The system has a means for transferring biomolecules held in proximity to the immobilized pH gradient member to at least one separation matrix.

In another embodiment, there is a method for two-dimensional electrophoresis. The method includes the steps of using an elongate immobilized pH gradient member to isoelectrically separate a macromolecular mixture; transferring the elongate member to a parallel electrophoresis cassette so that different regions of the member contact separation matrices held within the cassette; and applying an electric field to cause migration of biomolecules from the member into at least one matrix.

In another embodiment, there is a device for performing parallel electrophoresis. The device includes a support member adapted a hold a cassette having a plurality of parallel spaced apart electrophoresis gels. The support member has a lower electrode; an upper electrode retractably positionable against the cassette; and a safety lid adapted to prevent a electric shock hazard condition.

In related embodiments the device includes a plurality of optical detectors adapted to generate a plurality of electropherograms derived from samples electrophoresed in the gels.

In another embodiment a combined electrophoresis and blotting assembly includes an electrophoresis gel and a blotting membrane adjacent the gel. The blotting membrane is coated with a release agent so as to allow facile separation of the membrane and the gel after use in an electrophoresis and a blotting process.

### BRIEF DESCRIPTION OF THE DRAWINGS

The foregoing features of the invention will be more readily understood by reference to the following detailed description, taken with reference to the accompanying drawings, in which:

FIG. 1a is a schematic perspective view of a gel-membrane sandwich structure;

FIG. 1b is an exploded view of a casting frame for creating the sandwich structure of FIG. 1a;

FIG. 2 shows a flow chart of a method for producing a gel-membrane sandwich structure;

FIG. 3 shows a flow chart of a method in accordance with an embodiment of the invention that employs both chemical tensioning and membrane blocking;

FIG. 4a shows a perspective view of an assembled sandwich electrophoresis/blotting assembly;

FIG. 4b shows an exploded, perspective view of the assembly of FIG. 4a;

FIG. 5 shows an exploded, perspective view of an assembly employing spacers;

FIG. 6 shows a flow chart for a method of sequential electrophoresis and electroblotting;

FIG. 7 shows a benchtop apparatus for performing electrophoresis and electroblotting;

FIG. 8 shows a block diagram layout of the benchtop apparatus of FIG. 7;

FIG. 9 schematically shows an embodiment that is a cassette for holding multiple gels;

FIG. 10 schematically shows the loading ports of a cassette;

FIG. 11 schematically shows a cross section of an electrophoretic gel;

FIG. 12 schematically shows a rack for holding multiple cassettes;

FIG. 12b shows a representation of the rack of FIG. 12, with cassettes and a lid;

FIG. 13 schematically shows an optical arrangement for detecting molecules in a gel;

FIG. 14 schematically shows an optical arrangement for detecting molecules in a gel having multiple optical elements;

FIG. 15 schematically shows an array of gels attached to a membrane;

FIG. 16 schematically shows a template for gel band excision;

FIG. 17 schematically shows array of gels attached to a membrane overlayed on a template for gel band excision;

FIG. 18 schematically shows a membrane attached to an electrophoresis gel;

FIG. 19 schematically shows a side-view of an electrophoretic gel surrounded by a curved membrane;

FIG. 20 schematically shows an electrophoretic gel with a sample collection chamber.

FIG. 21 schematically shows a curved-path electrophoretic gel with two branches;

FIG. 22 schematically shows a curved-path electrophoretic gel with three branches;

FIG. 23 schematically shows a capillary for the collection of samples;

FIG. 24 schematically shows an arrangement for collection of samples into a capillary;

FIG. 25 schematically shows a member for use in isoelectric focusing;

FIG. 26 schematically shows a member for use in isoelectric focus atop a cassette;

FIG. 27 shows is a perspective view of an instrument for performing parallel electrophoresis in accordance with embodiments of the invention;

FIG. 28 shows a close-up view of a cassette holding area of the instrument of FIG. 27;

FIG. 29 shows the cassette holding area of the instrument of FIG. 27 and a grate extending over the cassettes;

FIG. 30 shows the instrument of FIG. 27 with a top in a closed position;

FIG. 31 shows a data readout in accordance with an embodiment of the invention.

### DETAILED DESCRIPTION OF SPECIFIC EMBODIMENTS

Definitions. As used in this description and the accompanying claims, the following terms shall have the meanings indicated, unless the context otherwise requires.

As used herein, the term “microplate” shall mean a receptacle having an array of vessels spaced on a 2-dimensional grid for holding 24, 96, 384, 1536 or larger number of samples. Examples of microplates include but are not limited to those that conform to standards set by the Society for Biomolecular Screening (www.sbsonline.org). Microplates are also referred to as “microtiter plates”.

As used herein, the term “microplate spacing” shall mean center to center spacing for a 2-dimensional microplate grid that is an integral fraction or multiple of about 9 mm.

Combined Electrophoresis and Blotting.

Illustrative embodiments of the invention relate to methods and devices for more efficiently performing electrophoresis and electroblotting. In an embodiment, a sandwich structure includes an electrophoresis gel affixed to a blotting membrane. In a related embodiment, a gel casting and/or running frame is used to hold the sandwich. In the further related embodiment there is a method and composition that allows separation of the gel and membrane after performing a combined electrophoresis and electroblotting operation so as to allow further operations to be performed individually upon the membrane, gel or both. In a further related embodiment, a uniform electrophoretic field is created by surrounding the sandwich structure using an insulating fluid; the insulating fluid is then swapped for a conducting fluid to allow application of an electroblotting field. In yet a further embodiment, an apparatus automatically manages fluid exchange and actuation of electrophoresis and electroblotting electrodes. In another embodiment, a plurality of parallel cavities are provided for holding multiple gels or gel membrane sandwiches.

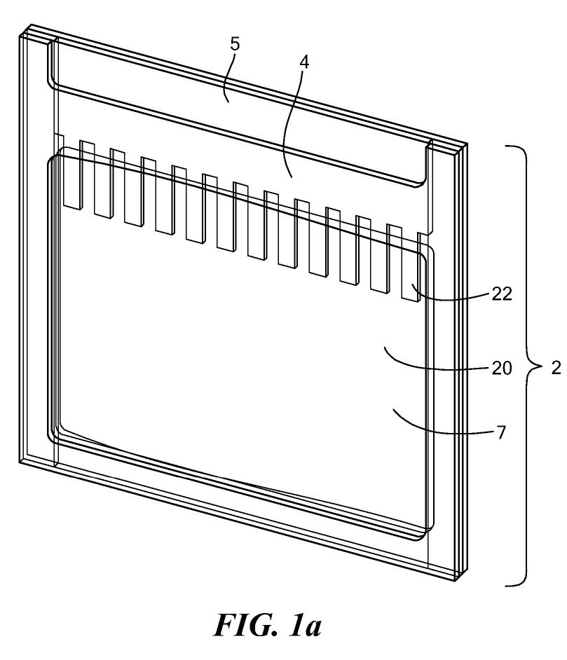

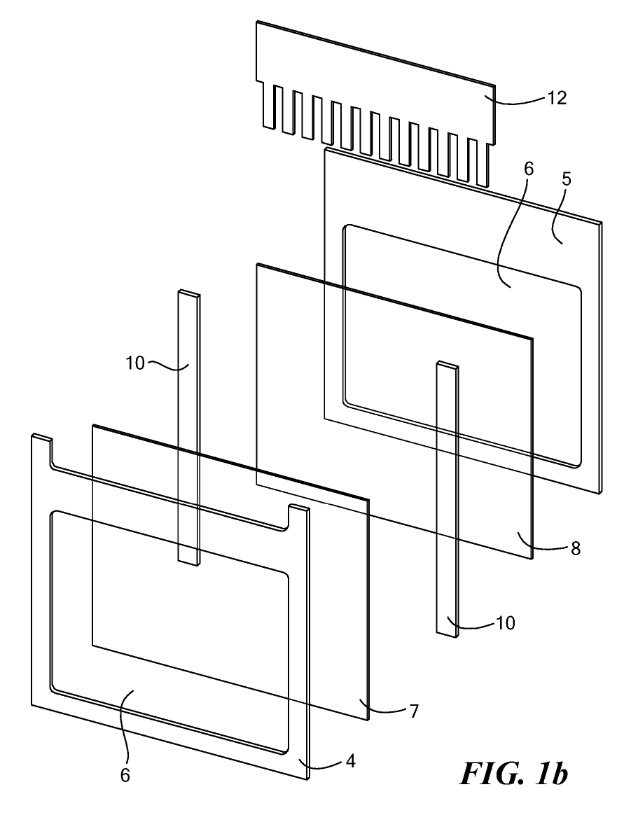

FIG. 1a is a schematic perspective view of a gel-membrane sandwich structure 2. FIG. 1b shows the structure in an exploded view, without the gel; the components shown in FIG. 1b may be used for casting a gel to create a sandwich 2. The sandwich structure of FIG. 1a may be used to perform a sequential electrophoresis and electroblotting operation. Samples are loaded in the wells of a gel 20 formed by a comb 12. The gel 20 is contacted with an electrophoresis buffer. An electric field is oriented in the plane defined by the gel to electrophoretically separate analyte molecules in the sample. A second, orthogonally oriented electroblotting field is then applied to drive the electrophoretically separated molecules to a blotting membrane 8, where the molecules may collect for further analysis. Alternately, the blotting step may be performed by a non-electrophoretic method, such as wicking.

In the sandwich 2, the gel 20 (e.g., an agarose or polyacrylamide gel) is affixed or otherwise held adjacently to at least one blotting membrane 8. In a preferred embodiment, the gel is also held adjacent to a conductive membrane 7 that is electrically permissive to an electroblotting field. The membranes 7, 8 may each be affixed so as to span windows 6 in a first frame 4, and a second frame 5. As shown in FIGS. 1a and 1b, the conductive membrane 7 is attached to the first frame 4 and the blotting membrane 8 is attached to the second frame 5, however, the orientation may be switched. The sandwich may be created by casting the gel 20 directly between the two membranes 7, 8 and is contained by lateral spacer strips 10. At least on of the frames may be notched out for sample-loading access.

The blotting membrane 8 may comprise, among others, nitrocellulose, nylon, polyvinylidene fluoride (PVDF) or derivatives of these, and generally binds molecules transferred to it from the gel 20 to facilitate subsequent analysis. For example, as is known in the art, PVDF membranes may be used for Western blotting applications. The pore size of the membrane may vary depending on the application; for example, it may be an average of 4.5 nm. The conductive membrane 7, may be composed of an identical material, but need not be, since for many applications, it is not required to have affinity for analytes. The conductive membrane 7 may be a woven or unwoven fibrous mesh, including a polyester mesh. In an alternate embodiment, the conductive membrane 7 may be omitted.

In an embodiment, the membranes 7, 8 are solvent-welded to their respective frames 4, 5. At least a surface of the frames 4, 5 may be constructed from a polymeric material. To solvent weld the membranes to polymeric frames, the membranes and frames are contacted in the presence of a solvent that will dissolve a portion of the surface of the polymer. The solvent and polymer will tend to penetrate the membrane's pores. As a result, when the solvent evaporates (e.g., in air, vacuum or heated conditions) the membrane will be affixed to the frame. As described below, the solvent welding may be performed in such a way as to chemically tension the membranes.

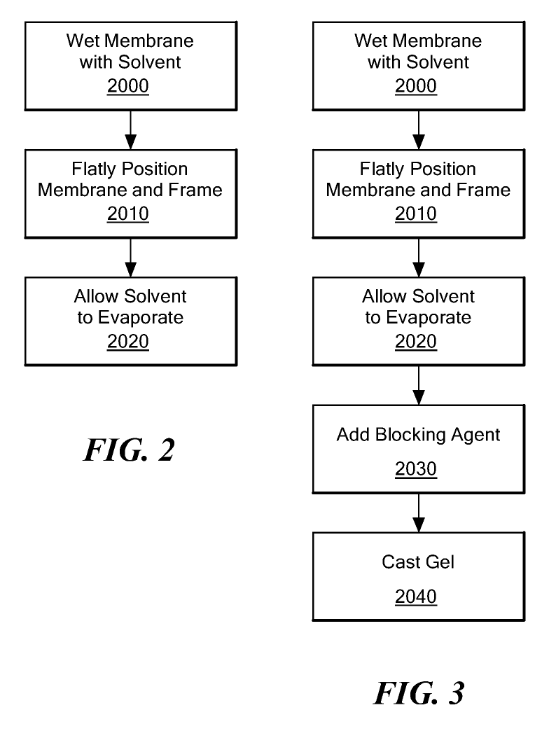

FIG. 2 shows a flow chart of a method for producing a gel-membrane sandwich structure in accordance with an embodiment of the invention. Certain types of porous membranes will swell in the presence of some solvents, and this property can be used to chemically tension the membrane across the frame. First, the membrane is wetted with the welding and tensioning solvent (step 2000). The solvent is chosen to cause lengthwise and widthwise swelling of the membrane as well as dissolving of the polymer frame. The solvent-wetted, swollen membrane is flatly positioned against the frame 5, and across the window 6 of the frame (step 2010) so that a border region of the membrane contacts the polymeric surface of the frame. (Alternately, the membrane may be positioned across the window 6 and then wetted). The solvent is allowed to evaporate (Step 2020). A solvent-weld will first form between the membrane and the frame and then the membrane will shrink to its original un-swelled state. To effectively tension the membrane, the weld should be formed prior to the majority of the shrinkage, or before the membrane has dried enough to initiate shrinkage. Typically, the solvent will evaporate from the edges of the membrane inward to first harden the weld, and then shrink the membrane. Resultantly, the membrane is stretched across the window 6 in a process of chemical tensioning.

Examples of welding and/or tensioning solvents include methyl ethyl ketone (MEK), acetone, and Weld-On™ (manufactured by IPS Corporation, Compton, Calif.), or combinations thereof. The degree of tensioning may be adjusted by adjusting the solvent composition. For example, acetone may cause more swelling than MEK, or Weld-On-4, and correspondingly higher tension. Different solvent compositions also evaporate at different rates. In an example, a mixture of MEK and Weld-On-4 is used to afford an intermediate degree of swelling and rate of shrinkage. The polymeric frame material may be, for instance, styrene acrylontitrile (SAN), polystyrene, polyethylene tetraphthalate (PET, PETE, PETP, or PET-P), or polyethylene tetraphthalate glycol (PETG). Since the welding process depends on the capability of the solvent to partially dissolve the frame, the choice of solvent should be matched to the frame material.

After performing electrophoresis and electroblotting, an experimentalist may desire to separate the membrane from the gel. For example, blotting with probes (e.g., Western, Southern, or Northern blotting) typically requires separation of the membrane from the gel because analysis may be impaired if the probes nonspecifically bind to the gel. However, if the gel 20 is cast in the presence of the membrane 8, the gel 20 may permeate the membrane 8 pores and render separation of the gel 20 and membrane 8 impracticable. To overcome this problem, an embodiment of the invention uses a coating or blocking agent to maintain separability of the membrane 8 and the gel 20. If a polymerized gel 20 is to be polymerized in the presence of the membrane 8 (e.g., a polyacrylamide gel), the blocking agent should be chemically permissive of the polymerization process. In addition, the blocking agent should effectively allow separation of the gel and the membrane. Gel polymerization may proceed in the presence of a membrane wetted with certain inert oils, such as mineral oil, and possibly silicone fluids, or certain polymer solutions such as methanol solutions of polyvinyl alcohol, but at least under some conditions, these may not permit subsequent separation of the gel and the membrane. Non-electrically conductive oils may also interfere with electroblotting.

However, if the membrane is treated with certain hydrophilic polymers polyvinyl acetate solution prior to polyacrylamide gel polymerization, the gel will polymerize and yet be readily separable from the membrane after electrophoresis. Similar results may be obtained if the membrane may be wetted with a polythelyene glycol solution (e.g., PEG 8000) or a starch solution (e.g., a 1% solution) and then dried prior gel polymerization. The solutions may be applied to the membrane in a variety of ways including wicking, spraying, dipping, painting or spin coating. The solutions may be aqueous or organic (e.g., methanolic) depending on the solubility of the blocking agent. If only one side of the membrane is coated, than that side should face the gel. It is possible that the coating materials allow release by retarding intrusion of the pre-polymerized acrylamide solution into the membrane.

FIG. 3 shows a flow chart of a method in accordance with a specific embodiment of the invention which employs both chemical tensioning and membrane blocking. First, the membrane is chemically tensioned and solvent welded to the frame (steps 2000-2020). Then, a aqueous or organic solution of blocking agent is dispensed onto the membrane (step 2030) and, optionally, dried. Then, a gel solution is added in a gel casting frame to polymerize or set a gel that is adjacent to the blotting membrane (step 2040).

In an alternate embodiment for positioning a membrane 8 adjacent to a gel 20, the membrane is held against the gel 20 by an affixation gel. For example, the gel 20 may be a polyacrylamide gel and the affixation gel may be an agarose gel. The resulting gel-membrane-gel sandwich may be produced using the solid gel-casting structures of FIG. 1b. For example, a membrane 8 may be sized to be small enough to fit within the window of the first frame 4 or the second frame 5. The frame is placed over the gel 20, and, the membrane 8 is then wetted with an aqueous solution and placed within the window, so as to flatly contact the gel 20 without trapping air bubbles, which are nonconductive and may interfere with blotting. The affixation gel is then cast over the blotting membrane 8 so as to fill the window 6. For example, a molten agarose solution may be dispensed into the window 6 and allowed to harden. Accordingly, when the gel has set, it will trap the blotting membrane 8 against the gel 20. An additional layer of moisture-retaining nonconductive material (e.g., a polyester or polycarbonate film) may then be placed atop the affixation gel. Optionally, or in addition, a thicker insert, such as a plastic component may be inserted into the window 6. A second insert may be used to cover the conductive membrane, if used. Electrophoresis may be accomplished with the inserts in place, with nonconductive film on both sides of the gel 20, or in the presence of an electrically insulating fluid, as described below. To perform electroblotting, any nonconductive layers (i.e., inserts and/or films) are removed and the gel is exposed to a transverse electroblotting field. The nonconductive layers may include features designed for ease of removal, e.g., slots or perforations which may be grabbed with a suitable tool. After electroblotting, the blotting membrane 8 may be removed by peeling away the affixation gel and blotting membrane 8 away from the gel 20. Alternately, an affixation gel may also used between the gel 20 and the membrane 8. A thin layer of agarose is spread over gel 20 and then the membrane 8 is applied (without air bubbles). Optionally, a second layer of gel (agarose or other) is overlaid on membrane 8 and then sealed using non-conductive moisture-retaining material.

In a further alternate embodiment, the blotting membrane 8 is held against the gel by a highly porous mesh, woven material or the like. A polyester mesh may be used. The mesh may be welded to a frame (4,5). For example, a polyester mesh may be solvent welded (and may be chemically tensioned) to a plastic frame, or welded with a heat gun. The frame and mesh are then used to sandwich a membrane 8 against a gel 20. In a specific emebodiment, a gel 20 is sandwiched on one side by a mesh and a Myler® film, and on an opposing side by a blotting membrane 8, a mesh, and a Mylar layer mounted on a frame. As in the preceding embodiment, thicker plastic inserts may also be used.

Using a thin insert or plastic film to cover the cassette 100 has the advantage of allowing for more efficient heat transfer than is achieved using traditional thicker electrophoresis cassette coverings such as glass plates. The film or insert should, however, retain sufficient dielectric properties to allow for effective electrophoresis.

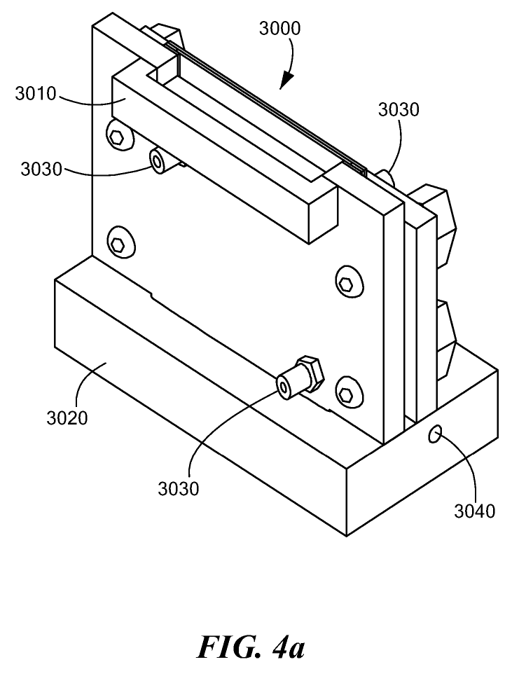

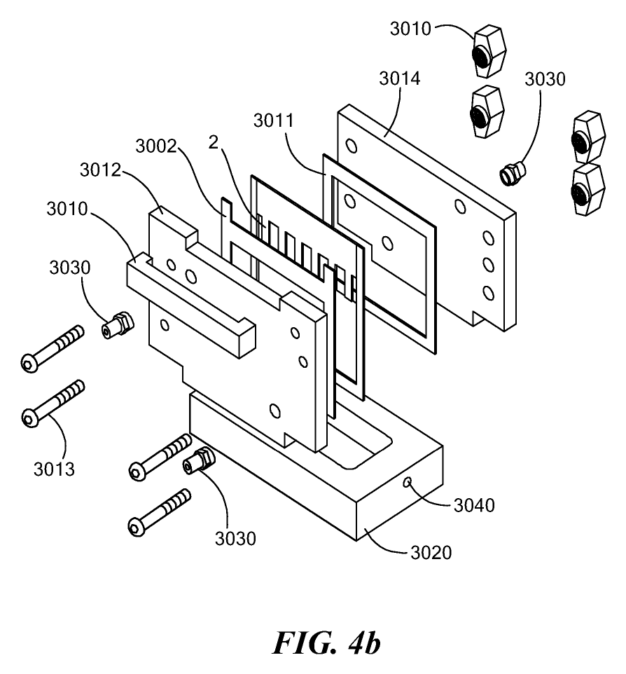

FIG. 4a shows a perspective view of an assembled sandwich electrophoresis/blotting assembly 3000. FIG. 4b shows an exploded view of the assembly 3000. A gel-membrane sandwich 2 is sealingly spaced-apart from a front plate 3012 and a rear plate 3014 by two gaskets: a front gasket 3002, with an upper sample-loading notch, and a rear gasket 3011. Each gasket (3002, 3011) has a window region, comparable in size to the window 6 of the sandwich 2, thus forming front and rear electroblotting buffer chambers when assembled. Screws 3030 and nuts 3010, or other suitable clamping arrangement may hold the front plate 3012, front gasket 3002, sandwich 2, rear gasket 3011 and rear plate 3014 together. The clamped-together structure may be supported by a base 3020. Electrophoresis buffer may be added to an upper reservoir 3010 formed by the front plate 3012 and in the base 3020. Electrodes (e.g., platinum wire or plates) may be positioned within the base 3020 and in the reservoir 3010 to effect electrophoresis when switched on. A hole 3040 in the base 3020 allows insertion of a wire electrode (or alternately/additionally, the exchange of buffer). Additional electroblotting electrodes for electrotransfer may be implanted in the front and rear plates (3012, 3014); when switched on, the electroblotting electrodes will cause current flow through the electroblotting chambers formed by the gaskets (3002, 3011) and through the sandwich 2 to cause analyte molecules to travel to the blotting membrane 8. Ports 3030 may be provided for the exchange of buffers in the electroblotting chambers formed by the gaskets (3002, 3011). The front plate 3012 and front gasket 3002 may be notched-out to allow access to the sample wells (e.g., with a pipette tip).

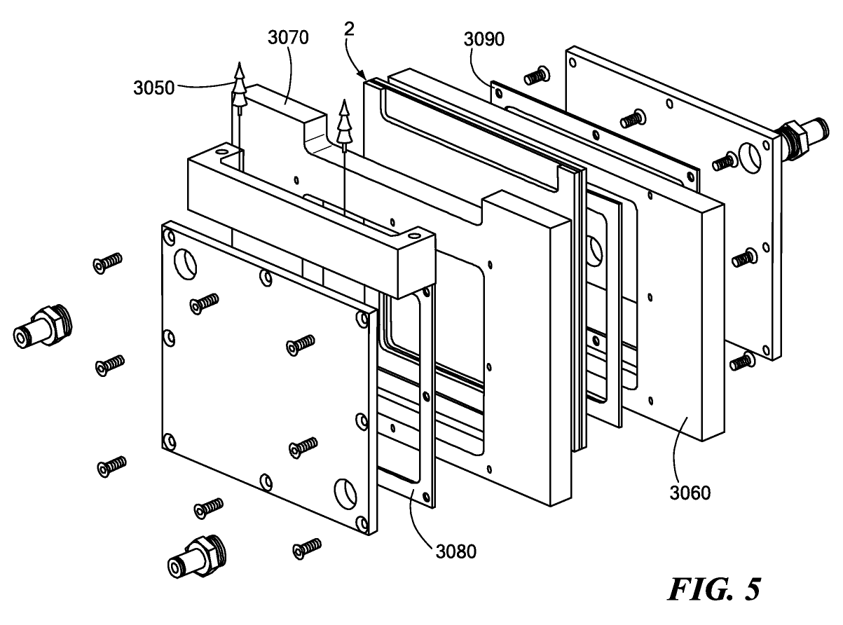

In an alternative electrophoresis/blotting assembly 3000, shown in the exploded perspective view of FIG. 5, the front and rear electroblotting chambers are expanded and allow for more buffer to be held therein. The electroblotting chambers are expanded by including front and back windowed spacers 3060 and 3070, along with additional front and back windowed gaskets 3080 and 3090, respectively. Electrophoresis buffer adaptors 3050 allow facile connection of tubing for electrophoresis buffer exchange.

In conventional electrophoresis, the gel is usually sandwiched between two insulating plates, often made of glass. In an experiment, protein molecular weight size standards were electrophoresed in a gel-membrane sandwich 2 with the membrane in an uninsulated state. In a control experiment the membranes were covered with an insert composed of electrically insulating material that filled the windows 6 of the sandwich 2. It was found that lower molecular weight protein bands were lost when the insert was not used, but were retained when the insert was used.

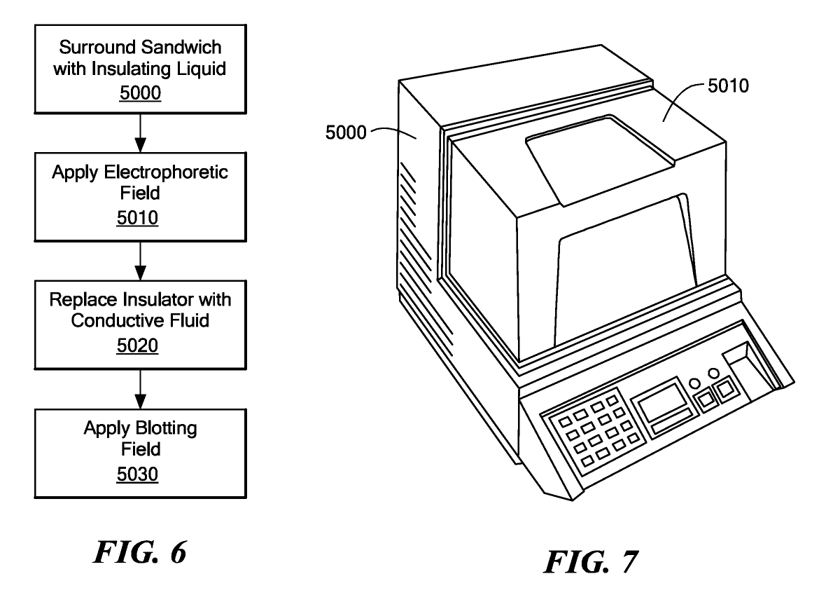

Therefore, to prevent loss of lower molecular weight biomolecules, a user may use an insulating material in an electrophoresis step, remove the material, introduce electroblotting buffer, and perform the electroblotting step. However, FIG. 6 shows a flow chart for a method of sequential electrophoresis and electroblotting using this principle, but substitutes an insulating fluid for the insulating insert, thus providing a less labor intensive and more automatable result in accordance with an embodiment of the invention. First, the sandwich 2 is surrounded by an insulating fluid (step 5000). For example, insulating fluid may be introduced into the side chambers of an electrophoresis blotting assembly 3000 formed by windows in the gaskets and/or spacers (Items 3060 and 3070 of FIG. 5) through corresponding appropriately positioned ports. Suitable electrically insulating fluids include perflourinated liquids such as Fluorinert™ from 3M Corporation. Other suitable perfluorinated fluids may include, perfluorodecalin and perfluorooctane. Additonally, some silicone fluids may be suitable. The fluid should be insulating enough to prevent loss of low molecular weight protein bands. A high degree of hydrophobicity is may also be important to prevent dessication of the gel 20. The fluid may have a dielectric strength of 40 kV at a 0.1 inch gap. The fluid may be cooled below ambient temperature in order to perform more rapid electrophoretic separations. Insulating fluid may be continuously perfused through the assembly 3000; however, a fluid should be chosen that does not freeze if perfusion is to be combined with sub-ambient cooling. After introducing the insulating fluid, the electrophoretic field is applied (step 5010) for a time sufficient to effect electrophoretic separation of the analytes in a sample. The insulating fluid is then replaced with a conductive electroblotting fluid (e.g., a buffer) and an orthogonal blotting field is applied for a time, and with a field strength sufficient to effect electroblotting. In an alternate embodiment, wicking is used instead of electroblotting for the blotting operation.

FIG. 7 shows a benchtop apparatus 5000 that accepts a electrophoresis/blotting assembly 3000 and automates one or more of the buffer exchange and electrode actuation processes needed to implement a combined electrophoresis and electroblotting procedure. The device may include a safety-lid 5010 that switches off high voltage sources when opened.

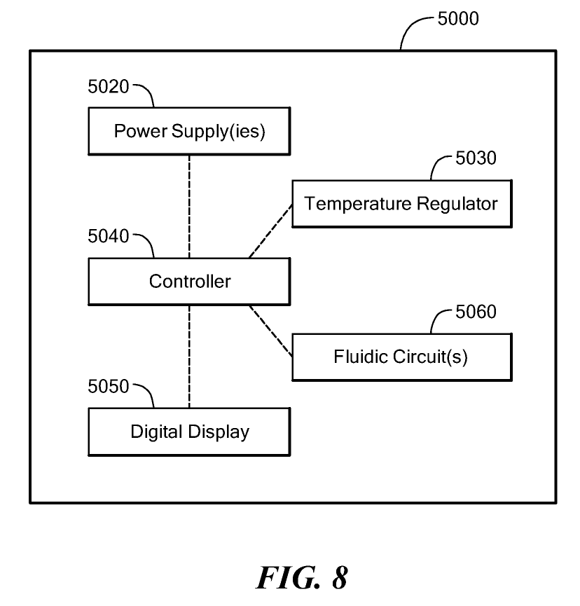

FIG. 8 shows a block diagram layout of a benchtop apparatus 5000 in accordance with an embodiment of the invention. The apparatus 5000 includes one or more power supplies 5020 for powering electrophoresis and electroblotting steps (e.g. adjustable 50-2000V DC sources). A fluidic circuit 5060 may be used to add or replenish buffers and/or insulating fluid. The fluidic circuit may employ one or more pumps, valves, reservoirs and conduits. One or more waste reservoirs may also be included. A temperature regulator 5030 either monitors the temperature, removes heat (produced due to Joule heating), or both. The temperature regulator 5030 may, for example, remove heat from one of the conduits or reservoirs in the fluidic circuit 5060. The temperature regulator 5030 may optionally employ a thermoelectric cooler, which may work in conjunction with a perfusion mechanism (e.g., a recirculating chiller and pump). A controller 5040 controls and monitors these processed and may output data on a display 5050 or other output device. The controller may also accept input parameters; e.g., power levels, run times, temperature settings, etc.

### EXAMPLE 1

### Electrophoresis and Electroblotting to Polyvinyl Acetate (PVAc)

To determine if protein transferred to membranes integrated during the gel casting process would be adversely affected by a PVAc coating, pre-stained protein standards and liver cell lysate were electrophoresed through a gel 20, and electrotransferred (electroblotted) onto the membrane 8 that had been pre-coated with PVAc prior to gel casting. The membrane was removed and probed with anti-HSP70 antibody for the detection of heat shock protein 70. The results demonstrated that PVAc coating did not adversely affect the immunoblotting process and the gel was recovered for further analysis of the transfer efficiency.

### EXAMPLE 2

Software and a controller are used to control the addition of the insulating fluids to the gel-membrane sandwich assembly 3000, which has an inlet and an outlet and thus acts as a flow cell. When the flow cell chamber(s) 3000 is full, the electrophoresis electrodes become engaged and electrophoresis takes place at the set voltage or current. Fluid is continuously circulated though the flow cell 3000 and cooled by a thermoelectric cooler. A digital display shows that the instrument is in the electrophoresis mode; and the time left until the electrotransfer is completed. At the conclusion of the electrophoresis step, the software switches off the electrophoresis electrodes; activates the transfer pump; removes the insulating fluid to its reservoir; switches valving to the conductive buffer reservoir position and begins to pump the conductive buffer to the flow cell chamber 3000. Once the flow cell chamber 3000 is filled, the software engages the electrotransfer electrodes to begin electroblotting transfer of protein or nucleic acids from the separating matrix to the transfer membrane. The digital display shows that the instrument is in the electrotransfer mode and the time left until the electrotransfer is completed.

### EXAMPLE 3

### Chemical Welding and Tensioning of Protein Blotting Membrane to Cassette Frames

Hydrophobic PVDF blotting membrane (Immobilon-FL, Millipore Corp., Bedford, Mass.) was cut to dimensions slighty larger than the windows of PETG front and rear frames (frames 4 and 5 from FIG. 1b). The cut membranes were wet and swollen by brief immersion in MEK (methyl ethyl ketone). The swollen membranes were spread onto a flat glass surface, and contacted with the frames with brief hand pressure. The frames were positioned so that the membrane completely overlapped the inside edges of the window. The frames, with attached PVDF membrane, were removed from the flat surface and the MEK was allowed to evaporate at room temperature. As the MEK evaporated, the membrane the shrank to provide a tight flat surface across the windows of the frames.

### EXAMPLE 4

### Coating of Protein Blotting Membrane with Hydrophilic Polymer: Starch

A 0.75% (weight/volume) suspension of starch in deionized water (Sigma Aldrich cat. #S9765-500G) was prepared by heating the mixture with constant stirring at 80 degrees C. for 2-3 hours. The mixture was allowed to cool, and formed a stable, translucent suspension. Approximately, 1 milliliter of the suspension was applied to the surface of a dry, tensioned, hydrophobic PVDF membrane-frame assembly described in Example 3. The dimensions of the membrane window were approx. 8 cm by 7 cm. The starch mixture was painted into a smooth layer using a disposable plastic foam brush, and allowed to dry at room temperature for 1-2 hours. Prior to assembly into a gel cassette, the membranes with wet briefly with methanol (100%), and then rinsed briefly with electrophoresis buffer to remove the methanol. The frames were then assembled into gel casting cassettes and used for gel casting. The membranes were not allowed to dry before gel casting.

### EXAMPLE 5

### Coating of Protein Blotting Membrane with Hydrophilic Polymer: Polyvinylacetate Adhesive

A 15% (weight/volume) suspension of a polyvinylacetate-based adhesive (PVA Size, Gamblin Artist Colors Co., Portland, Oreg.) was prepared in deionized water. The solution formed a stable, translucent suspension. Approximately, 1 milliliter of the suspension was applied to the surface of a dry, tensioned hydrophobic PVDF blotting membrane-frame assembly described in Example 3. The dimensions of membrane-covered window were approx. 8 cm by 7 cm. The PVAc mixture was painted into a smooth layer using a disposable plastic foam brush, and allowed to dry at room temperature for 1-2 hours. Prior to assembly into a gel cassette, the membranes with wet briefly with methanol (100%), and then rinsed briefly with electrophoresis buffer to remove the methanol. The frames were then assembled into gel casting cassettes and used for gel casting. The membranes were not allowed to dry before gel casting.

### EXAMPLE 6

### Use of Agarose to Install Non-Tensioned Protein Blotting Membrane into SDS-Protein Gel Cassette

Frames and spacers similar to those shown in FIG. 1b were used. A porous hydrophilic PVDF membrane (Durapore BVPP membrane, Millipore Corp., Bedford, Mass.) was tensioned onto the square frame (frame 6 in FIG. 1b). Protein does not bind to this membrane, and it is used merely to form an electrically-conductive gel boundary. A watertight, non-conductive plastic insert was installed into the window behind the membrane on this frame. The eared frame (frame 5 in FIG. 1b) was used without an installed membrane. A watertight insert was installed into the window of the eared frame. The two frames were assembled with spacers and comb into a cassette and a polyacrylamide SDS gel was cast in the cassette. After polymerization, the watertight insert was removed from the eared frame, thereby exposing the lateral surface of the polyacrylamide gel within the frame window. The cassette was laid on its side, with the exposed gel facing up. A hydrophobic PVDF blotting membrane (Immobilon FL, Millipore, Bedford, Mass.) was cut to fit within the frame window, wet with electrophoresis buffer, and placed directly against the polyacrylamide gel within the window. Care was taken to exclude air bubbles between the membrane and the gel. The remaining volume of space within the window above the blotting membrane was filled with a molten solution of agarose (1% weight/volume) in electrophoresis buffer, and allowed to harden at room temperature. After the agarose set, the window of the eared frame was covered with a watertight, nonconductive plastic material that sealed against the exterior surface of the frame using a pressure sensitive adhesive (Press'n Seal Freezer Sealable Wrap, Glad Products, Oakland, Calif.).

### EXAMPLE 7

### Use of Tensioned Mesh to Install Non-Tensioned Protein Blotting Membrane into SDS-Protein Gel Cassette

Frames and spacers similar to those shown in FIG. 1b were used. A porous hydrophilic PVDF membrane (Durapore BVPP membrane, Millipore Corp., Bedford, Mass.) was tensioned onto the square frame (frame 6 in FIG. 1b). Protein does not bind to this membrane, and it is used merely to form an electrically conductive gel boundary. A watertight, non-conductive plastic insert was installed into the window behind the membrane on this frame. The eared frame (frame 5 in FIG. 1b) was used without an installed membrane. A watertight insert was installed into the window of the eared frame. The two frames were assembled into a cassette and a polyacrylamide SDS gel was cast in the cassette. After polymerization, the insert was removed from the eared frame, thereby exposing the lateral surface of the polyacrylamide gel within the frame window. The cassette was laid on its side, with the exposed gel facing up. A hydrophobic PVDF blotting membrane (Immobilon FL, Millipore, Bedford, Mass.) was cut to fit within the frame window, wet with electrophoresis buffer, and placed directly against the polyacrylamide gel within the window. Care was taken to exclude air bubbles between the membrane and the gel. The blotting membrane was pressed securely against the polyacrylamide gel by installation of a smaller frame that fits tightly into the window of the eared frame. The smaller frame contains a window which is covered by a tensioned woven mesh of polyester fiber; this mesh presses the blotting membrane securely against the gel. To complete the cassette assembly, a watertight, non-conductive plastic insert is placed into the smaller insert behind the polyester mesh. This serves to seal the lateral gel surface during electrophoresis to separate the proteins.

Parallel Electrophoresis/Blotting

Other embodiments of the invention provide a parallel electrophoresis system (hereinafter “system”) suitable for either analysis or preparation of biomolecules. The system is typically arranged in the format of a microplate and may include cassettes in a strip format having individual gel elements of a number and spacing that corresponds to a row or column of a microplate. A rack vertically holds the cassettes and also may provide:
1) electrodes,
2) sources of electrophoresis buffer,
3) a heat removal mechanism,
4) one or more sensors for detecting the presence and/or position of a molecule within one or more gels, and
5) communication circuitry for communicating with a computer.
   A computer is typically provided to store and analyze sensor data and to control operation of an electrophoresis power source.

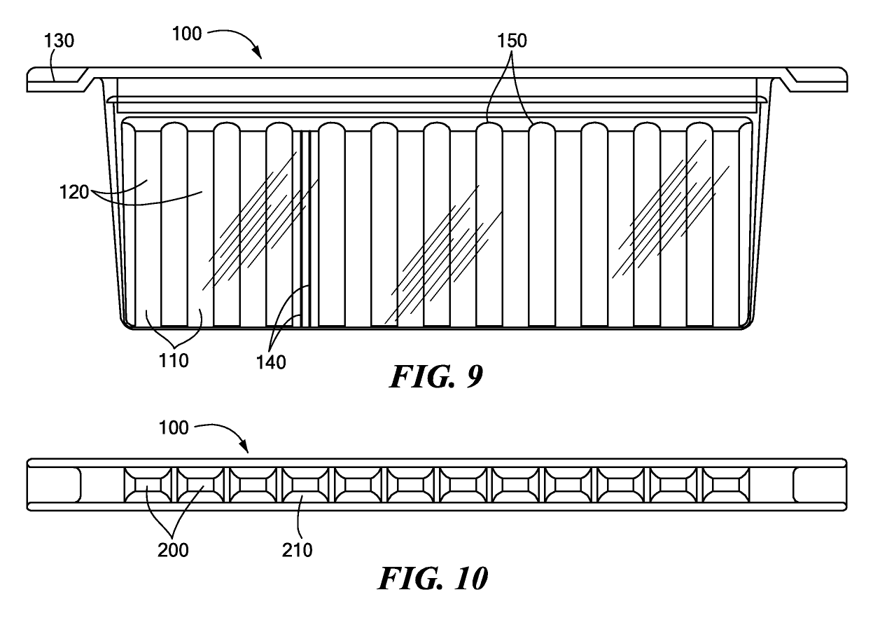

FIG. 9 show a side view of an embodiment that is an electrophoresis strip cassette 100 having elongate finger-like projections 120 for holding multiple electrophoretic gels 110 in a side-by-side arrangement. The projections 120 may all be integral to the cassette structure. Alternately, some or all of the projections may be held in place by intervening gels 110. The strip may be provided to an end-user with pre-cast gels, or the user may cast their own gels in the strip. A mold may be used to assist in the forming of the gels 110 within the cavities of the cassette 100. The gels are typically individual, independent, discontinuous structures and, as a result, molecules will not typically migrate or diffuse from one gel to another. The upper ends 150 of the projections 120 may extend above the cast gels 110 and define regions which, when bounded underneath by a gel, define individual, isolated sample holding wells. Tabs 130 are provided for easy gripping and handling of the strips and for aiding in insertion into and positioning in a rack (described in more detail below). The cassette 100 and projection 120 are typically composed of an inexpensive, electrically insulating material such as an injection-molded plastic. The plastic may be thin enough to allow efficient heat conduction from the gels 110. The cassette For example, the cassette may be a disposable, injection-molded plastic part. The cassette may include one or more membranes adjacent to the gels, including a blotting membrane 8. The blotting membrane may be in place during electrophoresis, or added after the electrophoresis step and may be packages in a kit with the cassette 100. In an alternate embodiment, the cassette 100 has no projections 120 so that the cassette 100 define a single gel 110.

The gels 110 of each cassette 100 are typically made of agarose, polyacrylamide or other gel-forming material suitable for electrophoresis and may be uniform throughout or gradient gels. The gel 110 typically takes the form of a right rectangular prism. By performing simultaneous electrophoresis experiments on a sample in multiple gels of varying porosity, a greater degree of dynamic range may be obtained in the experiment and optimal electrophoresis conditions may be discovered concurrently with analysis. An even greater number of conditions may be explored by using multiple cassettes each having differing sets of compositions. Gradient gels have a gradient in separation matrix properties such that the porosity of the gel varies along an axis of a gel. Gels 110 within one or more cassettes 100 may be of the same or different chemical composition. For example, a cassette 100 may hold twelve gels spanning a range of polyacrylamide crosslink densities and a second cassette 100 may hold an additional 12 gels spanning a second range of crosslink densities. Gradients may be continuous or have regions of discrete (stepwise) matrix composition. Stacking gels may be used, i.e., gels having a lower porosity gel region above a higher porosity region.

In an embodiment, the projections are tapered to create correspondingly tapered gels. By tapering the gels, electrophoresis artifacts, such as curved bands (“frowns” or “smiles”) may be minimized. This may occur due to compensation for edge effects. Edges effects would not typically be as problematic for conventional gels, where samples are run farther from the edges than in embodiments of the present invention.

In a further embodiment, each gel may be subdivided. One of the gels 100 depicted in FIG. 9 is shown with additional subdividers 140 to allow for multiple electrophoresis experiments to be performed on a single sample added to a single gel 110. For example, a different agarose or polyacrylamide gel percentage may be incorporated into the different subdivisions to increase the dynamic range of experimentation. Alternately, gels may be manufactured (without subdividers 140) that are composites of multiple gel strips of varying chemical composition.

FIG. 10 schematically shows a top view of an embodiment of the invention, detailing sample ports 200 integral to the cassette 100. These ports 200 create separate apertures for access to multiple, laterally-arranged gels 110. The walls 210 of the sample ports 200 typically serve to guide liquid to individual gels 110 and may form sample-loading wells when bounded at the bottom by gels 110. The wells will hold liquid when the cassette 100 is held in a vertical, upright position. Alternately, the ports may serve only to guide the tip of a liquid handling instrument to wells formed by the projections 120 and a gel 110. The process of dispensing samples onto the gels 110 may be accomplished with a single or multichannel pipettor, or other suitable liquid handling device. Although dense sample loading buffers and gel indentations (such as may be formed by a comb during gel casting) may not be necessary for loading, these techniques may still be used. The wells and corresponding gels in the strip advantageously have a center-to-center spacing that is an integral fraction of about 9 mm, which is the spacing of a standard 96-well microtiter plate (e.g., a standard microplate as defined by the Society for Biomolecular Screening). As an example, the cassette 100 of FIG. 10 has 12 wells and 12 gels, corresponding to one of the eight rows of a 96-well microplate. An alternate embodiment has a similar structure having 8 ports 200 and 8 gels 110, corresponding to one of the 12 columns of a 96-well microplate. In yet other embodiments, the strips have 24, 48, 16 or 32 well and gel elements corresponding to a row or column of a 384 well or 1536 well microplate format. The cassette may be provided to the end-user with a protective tape covering the wells.

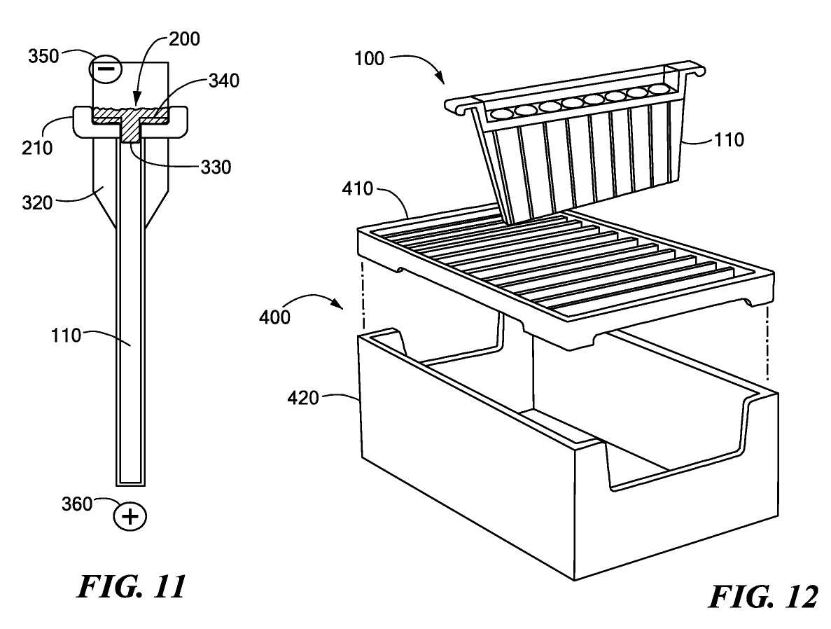

FIG. 11 schematically shows a cross-sectional view of a cassette 100 holding a gel 110. The walls 210 of a sample port 200, together with the upper terminus 330 of a gel 110, define a well that holds a sample 340. A buttress 320 gives structural stability to a linear array of ports 200. To resolve molecules within the sample, an electric field is applied by the electrical connection of an electrophoresis power source to an electrophoresis anode 360 and an electrophoresis cathode 350. During electrophoresis, negatively charged molecules such as nucleic acids or proteins complexed with anionic detergent typically travel toward the electrophoresis anode 360 at a rate that is dependent on the applied voltage, the sieving-properties of the gel, and the hydrodynamic size of the molecules. Among other things, the electrodes may be composed of inert metals, such as platinum wire. A wire pin, ring, or other metal structure may be inserted into the sample to serve as the electrophoresis cathode 350. Alternately, the electrophoresis cathode 350 may be built into the cassette 100. For example, a platinum ring may be attached to the inner perimeter of the walls 210 with an attachment lead connecting above the sample. The electrophoresis anode 360 may be in the form of a platinum wire in a buffer tank that is common to, and covers the lower terminus of, multiple gels 110. Alternately, each gel 110 may have its own electrophoresis anode 360 situated in a lower well (described in more detail below).

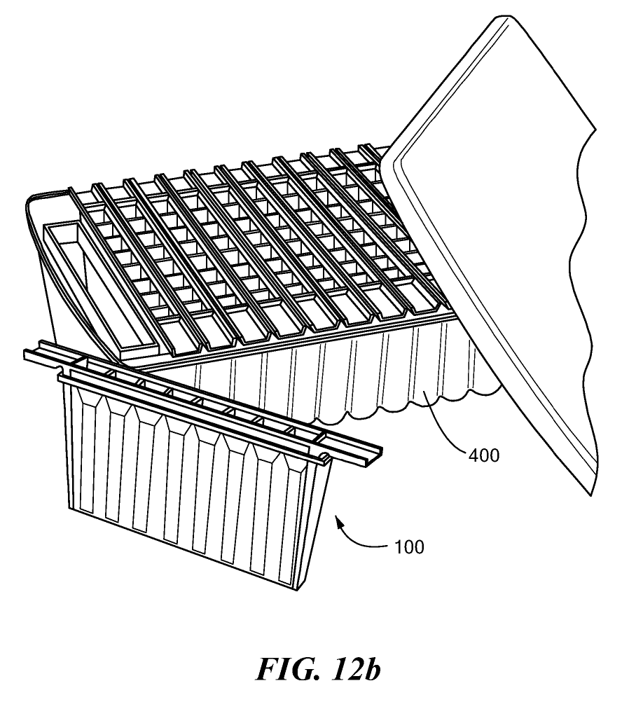

FIG. 12 schematically shows a rack 400 for holding multiple cassettes 100, such as those cassettes 100 discussed above. The rack may have an upper portion 410 and a lower portion 420. One or more cassettes 100 may be positioned in a cassette-holding rack 400 in an upright, vertical position suitable for loading and electrophoresis. The rack 400 can hold many different sized cassettes 100. For example, the rack 400 may hold:
A) 12 cassettes 100 of 8 gels 110 for a total of 96 gels 110,
B) 8 cassettes 100 of 12 gels 110 for a total of 96 gels 110,
C) 24 cassettes 100 of 16 gels 110 for a total of 384 gels 110,
D) 16 cassettes 100 of 24 gels 110 for a total of 384 gels 110,
E) higher or lower density microplate-compatible arrangements,
F) cassettes with non-SBS microplate spacing, or
G) cassettes with non microplate spacing.
   In illustrative embodiments, the rack 400 is configured to hold the cassettes 100 at a spacing that mimics a specific microtiter plate, such as a 96 well, 384 well, 1536 well, or other density microtiter plate. For example, 8 cassettes 100 having 12 gels 110 may be spaced 9 mm apart to give the overall geometry of a 96 well plate. One advantage of mimicking a standard microplate geometry is that the cassettes 100 may be placed in a rack and loaded from source microplates using a standard fluid handling robot. Another advantage is that either an 8 or 12 element multichannel pipettor (e.g., the Gilson Pipetman® Ultra Multichannel from Gilson, Inc., Middleton, Wis.) may be used to load the wells 200. FIG. 12b shows a representation of a rack 400 with cassettes 100, and a lid; the rack holds 96 gels in a 12×8 configuration with a standard 9 mm microplate spacing.

The rack 400 may have a top portion 410 for holding the cassettes 100, and a corresponding bottom portion for containing an electrophoresis buffer. Moreover, the rack may contain, among other things, electrophoretic buffer reservoirs, temperature control mechanisms, electrodes having leads to a power source (e.g., a DC supply capable of voltages in the range of 100V-1500V), optical components (e.g., scanning optical components with associated actuation elements). To provide a sufficient amount of room for these elements, the rack 400 may be expandable For example, the expandable rack 400 may include sliding or pivoting elements that join the cassettes 100 in an arrangement that allows their distance to be increased either manually or automatically.

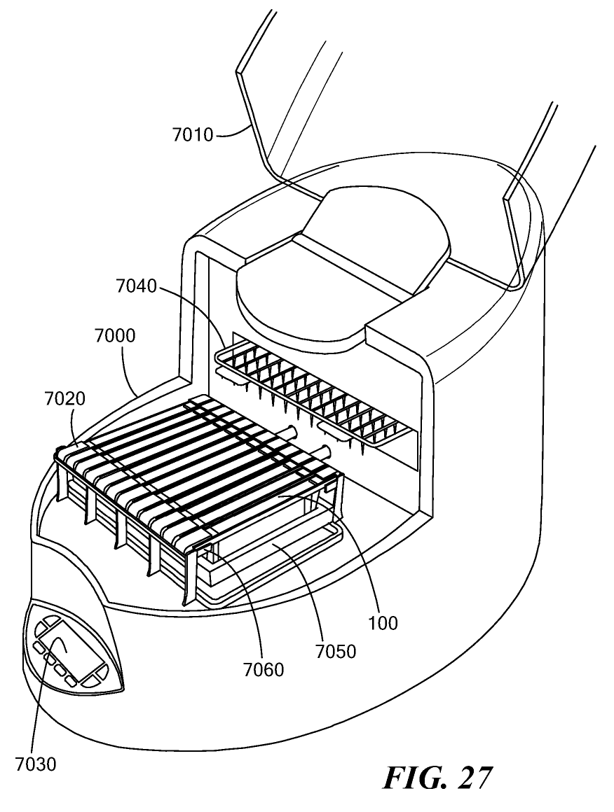

The rack 400 may include a cable or wireless data communication conduit (including WiFi or BlueTooth circuitry) for relay of control signals from a computer and upload of data from sensors included in the rack 400. Sensors may be used to measure temperature, fluid level, and electrophoretic progress via optical measurements. The rack 400 also may provide temperature control for cooling and/or heating of the gels 110 to allow for more rapid experiments through the dissipation of Joule heating. The buffer may be cooled by various means, including ports for connection to a re-circulating chiller or an attached electrothermal cooling device such as a Peltier cooler. The temperature control mechanism may also include a heater, which may be used to accomplish denaturing gradient gel electrophoresis (DGGE). The rack 400 further may include data and/or fluid connections for use in docking with a base-station having buttons, switches, displays or connections to a computer. Alternately, the rack 400 simply holds the cassettes 100 prior to use in an analytical instrument, one embodiment of which is described below with reference to FIGS. 27-31.

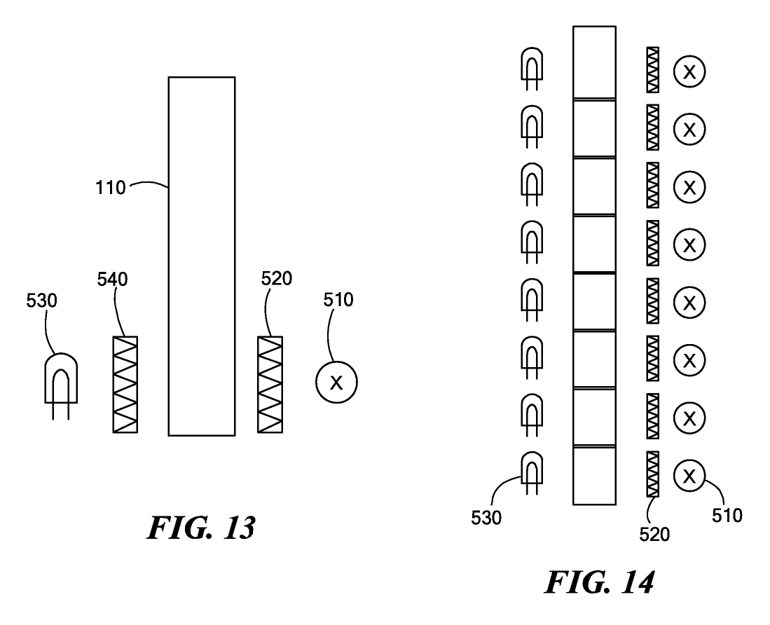

The optical components, which may be fixed or scanning optics, among other things, typically allow for absorbance or fluorescence measurements of molecules in the gels 110, and allow measurements that are positionally and/or temporally resolved. As shown in FIG. 13, each gel 110 illustratively has one or more optical elements. Each optical element may have multiple components, such as at least one light source 530, at least one detector 510, and at least one associated optional excitation or emission wavelength selecting devices 520 and 540 (e.g., colored glass or holographic interference filters). Detection of biomolecules may be accomplished by using the optical elements to detect native absorbance, such as ultraviolet absorbance or aromatic groups of the biomolecules, or fluorescence measurements to detect the fluorescence of biomolecules and/or associated dyes or stains. The optical components may be mounted directly in the rack, or may be remote and utilize light guides, such as fiber optics. If mounted directly in the rack, illustrative embodiments use water-resistant components.

FIG. 14 shows an embodiment of the invention having one optical element positioned near the bottom of each gel 110. This optical element includes an LED light source 530, an interference emission filter 520 to filter stray excitation light and a photodiode detector 510. The excitation and emission optics may be placed on the same side, or on opposite sides of the gels 110. Stray light noise may be minimized by positioning the excitation and emission optics on the same side of the gels 110 and aligning the angle formed between the incident light source and the detector at approximately 90 degrees. Light barriers, such as black plastic components, may be placed between cassettes 100 in the rack 400 to reduce optical crosstalk. Another way to reduce optical crosstalk is to acquire signals in an intermittent manner by switching the multiple excitation sources on at different times in a manner that avoids simultaneously illuminating adjacent gels. The optical elements operate to detect fluorescence from a molecule or molecular complex, such as double stranded DNA complexed to a dye such as SYBR Green (Invitrogen Corporation, Carlsbad, Calif.). In one analytical mode, some or all of the biomolecules may be electrophoresed past the detector and even off of the gel (thus giving improved dynamic range). The signal from the detector is logged by a computer over time and used to automatically determine when the run is complete (which may result in an automatic switching off of the power supply) and to create a visualization, such as a signal vs. time chart. The logged signal data may also be used for quantitative analysis of the data, such as determining the absolute or relative amounts of biomolecules present in the electrophoretic bands.

The optics may be capable of lateral resolution along the gel 110. For example, if multiple detecting elements such as could be provided by a CCD array are used, multiple lanes may be resolved within each gel 110. The multiple lanes of subdivided gels may be resolved in this manner. Alternately, the rack may be capable of scanning in a lateral dimension vertically by acquiring multiple images while moving the gels 110 or the detectors relative to each other.

The optics also may be capable of vertical resolution either by having multiple sensing elements along the length of the gels 110, or by scanning the detectors vertically by acquiring multiple images while moving the gels 110 or the detectors relative to each other.

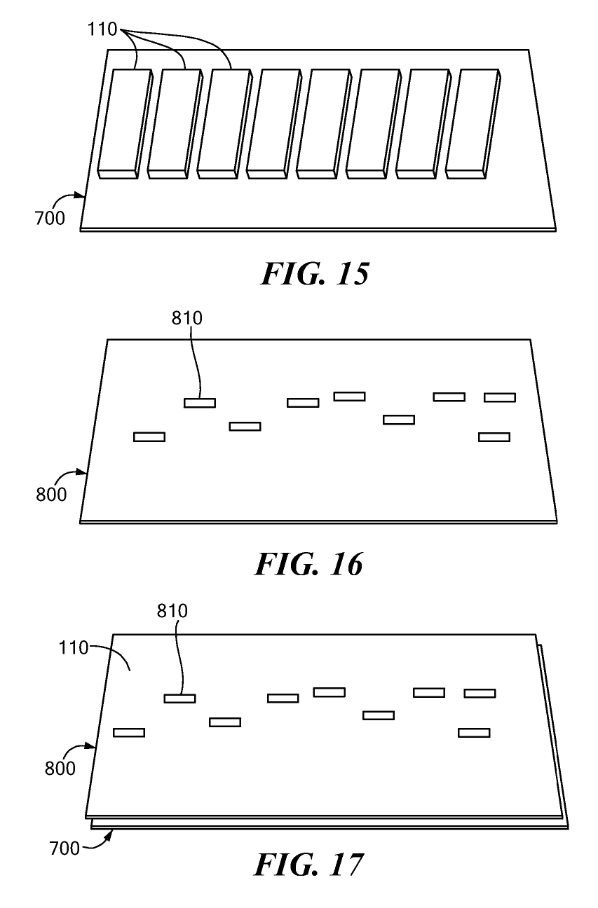

Prior to electrophoresis, some embodiments position a membrane adjacent to the gels to aid in recovery of the gels, or to perform a blotting operation, such as electroblotting. Use of a membrane for recovery is described with reference to FIGS. 15-17, while use of a membrane for use in electroblotting is described with reference to FIGS. 15-16.

FIG. 15 schematically shows an embodiment having gels attached to a removable, “peelable membrane 700.” The gels 110 are affixed to the peelable membrane 700 so that when the membrane is peeled away from the cassette 100, the gels are removed along with the membrane 700. Samples may then be recovered from the gels for storage or analysis, or the gels may be stored by freezing, drying or other archiving technique. The affixation of the membrane to the gels may be accomplished in a number of manners, such as by chemical or photochemical crosslinking during or after the formation of the gel. A weak, reversible adhesive may loosely affix the projections 140 and the membrane 700.

In many cases, it may be desirable to excise particular regions of the gel having one or more molecules of interest, such as a particular protein or nucleic acid band. In accordance with illustrative embodiments, the computer produces a template for excising such molecules. To that end, the computer may utilize user input and data related to the positions of biomolecules in a gel, either alone or in combination with data from standards in one or more reference gels, to print a excision template 800 having visual indicia 810, as shown in FIG. 16. For example, a combined electrophoresis and fluorescence detecting instrument may use fluorescent data acquired during an electrophoresis run to generate a template. FIG. 17 shows the gels 110 on the peelable membrane 700 aligned atop the template 800. The peelable membrane 700 and gels 110 illustratively are transparent so that the indicia 810 are visible through the gels 110. Reference features or markings may be provided on both the template 800 and peelable membrane 700 to aid in proper alignment. A razor blade or other cutting tool may then be used to excise the desired bands using the indicia 810 as a guide.

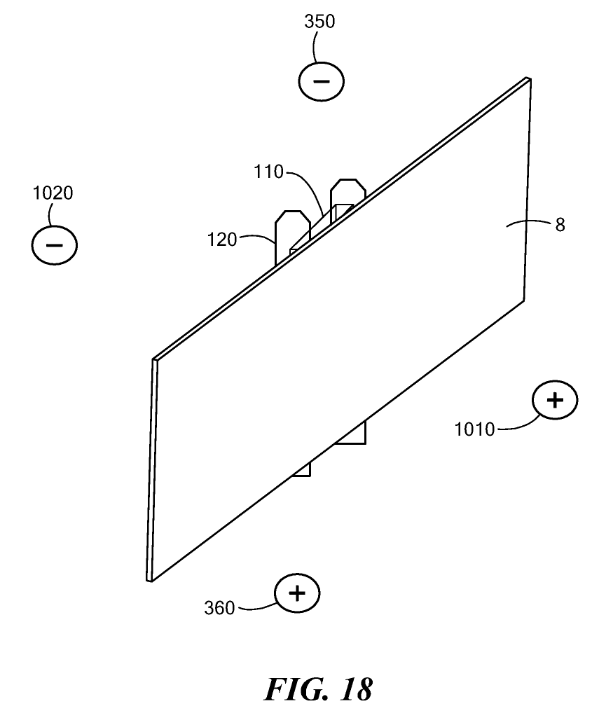

FIG. 18 shows an embodiment using a membrane 1000 for electroblotting. An electrophoresis cathode 350 and an electrophoresis anode 360 are first used to resolve biomolecules on a gel 110 held between dividing projections 120, while one or both sides of the gel 100 are reversibly attached or held against an electroblotting membrane 8. Following electrophoresis, the electrophoresis cathode 350 and electrophoresis anode 360 are switched off. Electroblotting electrodes, such as an electroblotting anode 1020 and electroblotting cathode 1010 are then switched on to drive desired biomolecules toward one or more membranes. Among other things, the electroblotting electrodes may be plate electrodes to provide a uniform electric field. The electroblotting membrane 8 may then be removed for additional analysis, such as UV crosslinking, Southern, northern or western analysis, or archiving. Biomolecules may be crosslinked to the membrane 8 while the membrane 8 is still in the cassette 100, or after removal of the membrane 8 from the cassette 100. In an embodiment. the peelable membrane 700 of FIG. 17 may be used in combination with an electroblotting membrane 1000 such as shown in FIG. 18; e.g., a peelable membrane 700 of one side of a gel 110 or cassette 110 and an electroblotting membrane 1000 on the opposing side.

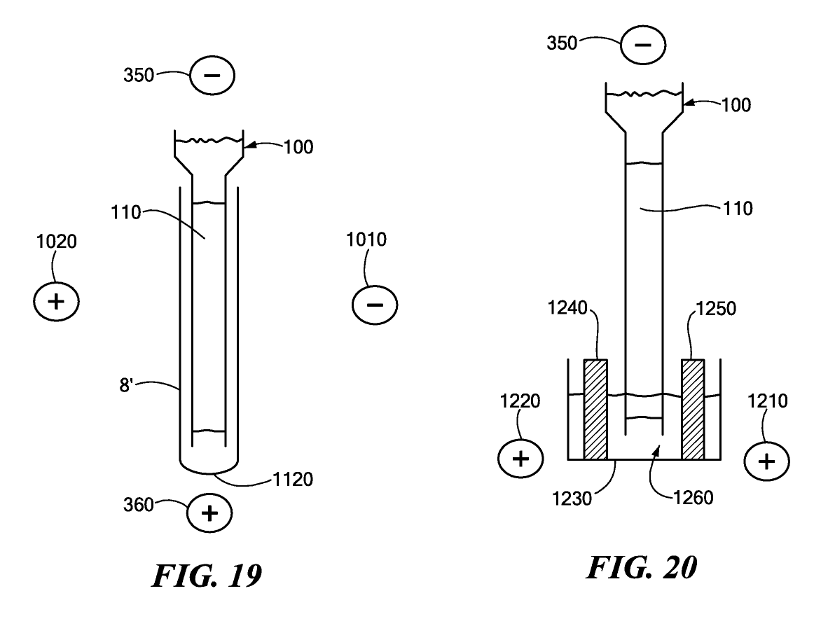

FIG. 19 shows a cross-sectional view of an embodiment of the cassette 100 in which a single electroblotting membrane 8′ wraps around and contacts both sides of the gel. In operation, smaller molecules may migrate off the bottom terminus of the gel 110 and may be trapped on, or migrate through, the bottom portion of the electroblotting membrane 1000, while larger molecules are resolved within the gel. After electrophoresis, the electroblotting electrodes are switched on, consequently driving positively charged molecules toward the electroblotting anode 1020 and negatively charged molecules toward the electroblotting cathode 1010. Thus, if both negatively charged and positively charged species are included in the sample (such as might occur in native protein electrophoresis), the single electroblotting membrane 8′ should have a region of adhered basic proteins and a region of adhered acidic proteins.

FIG. 20 shows an alternate embodiment having a cup 1230 for collecting electrophoretically resolved biomolecule fractions. The lower section 420 of the rack 400 has one or more cups 1230 that hold electrophoresis buffer 1260, which contacts the lower terminus of the gel 110. A liquid sampling device (such as the embodiment employing a capillary described in more detail below) may withdraw liquid from the cup 1200 with a timing that may be determined by a computer. The timing may be based on measurements of migration rates made by an optical detector 510. The cup may include a collection anode 1210. As desired molecules are eluted from the gel 110, care should be taken to prevent the molecules from being destroyed by the collection anode 1210. This may be accomplished, by lowering the electric field strength around the time of collection and/or surrounding the collection anode 1210 with a membrane or gel that is prevents passage of macromolecules, but permits the current-conducting flow of electrophoretic buffer salts.

Alternately, a waste anode 1220 may be switched on during the period of the electrophoresis run in which unwanted biomolecules are being eluted from the gel 110 as determined by optical measurement by detectors 510 and/or predicted based on timing. Unwanted biomolecules are thereby drawn to, and trapped in, a waste collection gel 1240, or travel through waste collection gel 1240 and thus, are destroyed by waste anode 1240. During periods in which desired molecules are predicted to be eluted based on optical measurement or timing, the collection anode 1210 may be switched on for a time sufficient to trap molecules in a sample collection gel 1250.

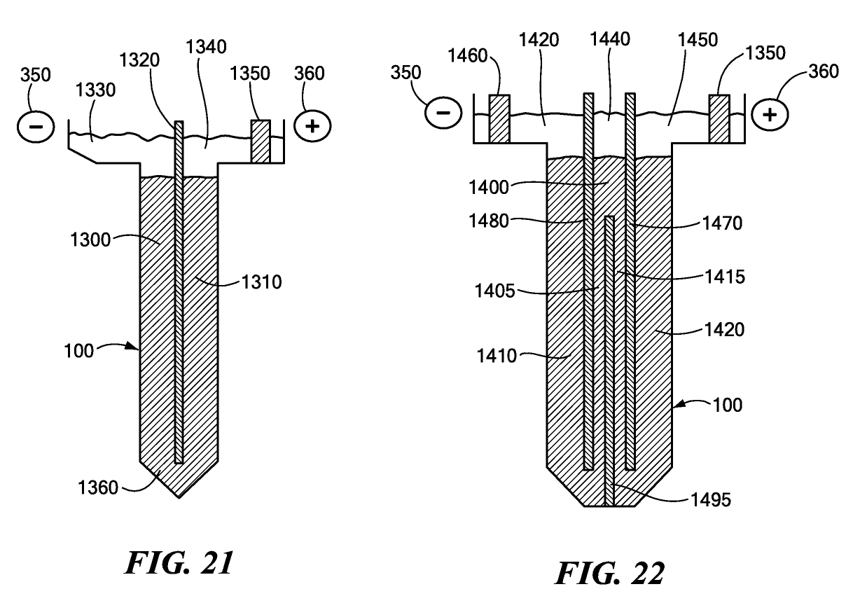

Alternate embodiments of the invention utilize curved-path electrophoresis. An embodiment using a curved electrophoresis path with a “u-shaped” bend is shown in FIG. 21. A divider separates an initial downward electrophoresis gel portion 1300 from an upward return gel portion 1310. A curved-path gel portion 1360 joins the downward gel portion 1300 and the return gel portion 1310. In use, a sample in buffer is added to a sample chamber 1330. A buffer added to a collection chamber 1340 at the upper terminus of the return gel portion 1310 is separated from the sample chamber 1330 by an insulating divider 1320. An electric field is applied using an electrophoretic anode 360 positioned in electrical contact with the collection chamber 1340 and an electrophoretic cathode 350 positioned in electrical contact with the sample chamber. The electric field causes migration of macromolecules from the sample chamber 1330 down the downward gel portion 1300, around the curved gel portion 1360 and up the return gel portion 1310. Under continued application of voltage, macromolecules arrive in the collection chamber 1340 where they may be removed by an appropriate liquid handling mechanism such as a pipette or sipper tube (described in more detail below).

An ion-conductive electrode barrier 1350 may be employed to prevent macromolecules from being damaged or destroyed at the electrophoretic anode 360. A similar barrier may be used in the sample chamber 1330 to protect molecules from the electrophoretic cathode 350, if desired. The barrier 1350 may be composed of a semipermeable membrane or gel. Alternately, the barrier 1350 may be a highly charged membrane. The membrane or gel may be in the form of a coating around the electrophoretic anode 360. If a gel barrier 1350 is used, a high-density gel should cause molecules to be retained in the sample chamber 1340 and not be ensnared in the gel itself Alternately, a lower density gel may be used and the gel recovered for further use. If desired macromolecules do become embedded in the barrier, the current may be temporarily reversed to back-elute the molecules from the barrier 1350. The current may be automatically paused based on the predicted or measured (e.g., via optical measurement of the gel 110 or chamber 1340) presence of desired macromolecules. Multiple u-shaped electrophoresis gels may be incorporated into a cassette 100.

A computer can advantageously control the on/off, pause, and reverse functionality of the power supply. A liquid handling instrument monitor, which may be a proximity detector, such as an infrared LED light source with a photodetector, may be employed to sense when a liquid handling instrument (such as a pipette tip of a pipette) has accessed the sample chamber 1340 and/or withdrawn sample. The liquid handling instrument monitoring function could also include a fluid level monitor in the collection chamber 1340 or a signal from a liquid handling robot or semi-automatic electronic pipette. When the monitor senses withdrawal of liquid, electrophoresis may switch from pause to resume.

After withdrawal of sample, it will often be necessary to add additional electrophoresis buffer to the collection chamber 1340. This may be done manually or with an automatic dispensing system. A level monitor in the sample chamber 1330 and/or collection chamber 1340 may be used to trigger automatic addition of buffer or to alert a user to a low buffer condition. Application of current may be automatically paused until additional buffer is added. Buffers may need to be periodically replaced during a run. The u-shaped gel is typically confined by and part of a cassette 100.

FIG. 22 shows another embodiment that utilizes curved-path electrophoresis with two “u-shaped” gel paths having multiple branches. The gel illustratively is confined by, and part of, a cassette 100. The gel has a downward branch 1400 connected via curved gel portions to a cathodic return branch 1410 and an anodic return branch 1420. An insulating median divider 1495 splits the downward branch 1400 into a downward branch 1405 and an anodic cathodic downward branch 1415 and causes the electric field to traverse the cathodic downward branch 1405, downward branch 1400, and an anodic downward branch 1415. A cathodic-side insulating divider 1480 and an anodic-side insulating divider 1470 maintain physical and electrical separation of the return branches. Electrophoresis buffer thus is held in three separate reservoirs: a cathodic chamber 1420, a neutral chamber 1440, and an anodic chamber 1450. An anode barrier 1350 and a cathode barrier 1460 protect biomolecules from the electrodes, as described above with reference to FIG. 21.

Among other things, gel of FIG. 22 is useful for native protein electrophoresis. In operation, a protein sample is typically mixed with an electrophoresis buffer and applied to the neutral chamber 1440. Electrophoresis buffer also is applied to the anodic chamber 1450 and cathodic chamber 1420. A DC voltage is applied via an electrophoretic cathode 350 and an electrophoretic anode 360 to cause charged protein molecules to migrate down the downward branch 1400. Basic, positively-charged proteins will begin to migrate down the cathodic downward branch 1405, around a lower terminus of the cathodic-side insulating divider 1480 and up the cathodic return branch 1410 toward the cathode 350, while acidic, negatively-charged proteins will begin to migrate down the anodic downward branch 1415, around a lower terminus of the anodic-side insulating divider 1470 and up the anodic return branch 1420 toward the anode 360. During the process, uncharged proteins will remain in the neutral chamber 1440. Eventually, basic proteins will reach the cathodic chamber and acidic proteins will reach the anodic chamber. Proteins may be collected, buffers exchanged, and the electrodes controlled as in the u-shaped embodiment described with reference to FIG. 21.

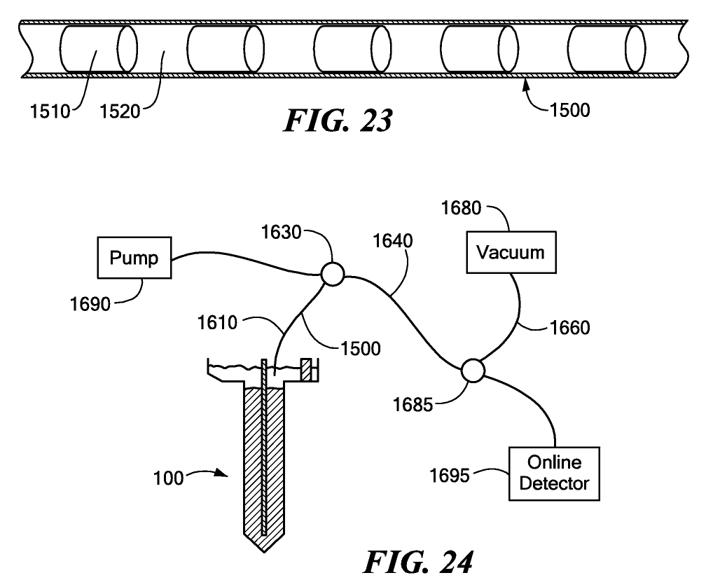

The rate of sample production by parallel operation of multiple gels 110 in one or more cassettes 100 may exceed the rate at which they may be analyzed or otherwise used. As a remedy, samples retrieved from the chambers of the various embodiments may be advantageously stored in a microplate, or other device, for further analysis or use. FIG. 23 shows an embodiment having a sipping capillary 1500 for storing electrophoretic fractions prior to use. The capillary 1500 may be a narrow bore plastic capillary tube of sufficient length to hold multiple samples 1510. The samples 1510 held in the capillary 1500 may be separated by a plug of air 1520 or immiscible liquid, such as an oil or volatile organic solvent. An automated, robotic system may position the input of the capillary 1500 in various sample chambers at appropriate times, as determined by optical measurements, to receive desired samples. Samples may be introduced into the capillary 1500 by application of a negative pressure at a distal end, and subsequently dispensed by applying positive pressure to a proximal end.

In illustrative embodiments, the capillary 1500 outputs to an online detector, such as mass spectrometer (e.g. with an APCI or ESI interface). For applications involving mass spectrometry, sample preparations steps are often needed. A desalting step is usually necessary to remove electrophoresis buffers that cause ion suppression and blockage of the mass spectrometer orifice. Enzymatic digestions may also be appropriate in some applications, such as the processing of protein samples for sequence-based identification and measurement. Sample held within the capillary 1500 should interface with a variety of online microfluidic sample preparation devices. One example of such a device is sold by Micronics, Inc. of Redmond, Wash. and uses laminar flow to extract small molecules from a liquid stream. Advion, Inc. of Ithaca, N.Y. commercializes a device that provides a miniaturized nanospray ESI interface. Additional on-line devices for sample processing prior to mass spectrometry include the RapidFire™ CX-MS (BioTrove, Inc of Woburn, Mass.) and Turbflow™ (Cohesive Technologies, Inc. of Franklin, Mass.).

FIG. 24 shows an embodiment having a capillary 1500 that feeds to an on-line analyzer, such as a triple-quadrapole or time-of-flight mass spectrometer having an atmospheric pressure chemical ionization (APCI), atmospheric pressure photoionization (APPI) or electrospray ionization interface. A sipper portion 1610 of the capillary 1500 is attached to a negative pressure source 1680 via a first valve 1630 and a second valve 1685. The proximal end of the sipper portion 1610 is positioned in a collection chamber of the cassette 100. The valves 1630 and 1685 are positioned to connect the sipper portion 1610 with the vacuum source so that a sample may be drawn into the sipper tube. Removal of the sipper portion 1610 from the collection chamber causes a plug of air to enter the sipper portion. The sipper portion 1610 is then placed in another sample chamber of cassette 100, or the sample chamber of a second cassette 100 in a rack 400. By this process, a series of samples separated by air plugs may be introduced into the sipper portion 1610, and air plugs may then be drawn into a holding portion 1640. The valves are then switched to allow a positive pressure source 1690 to drive samples to an online analyzer 1695.

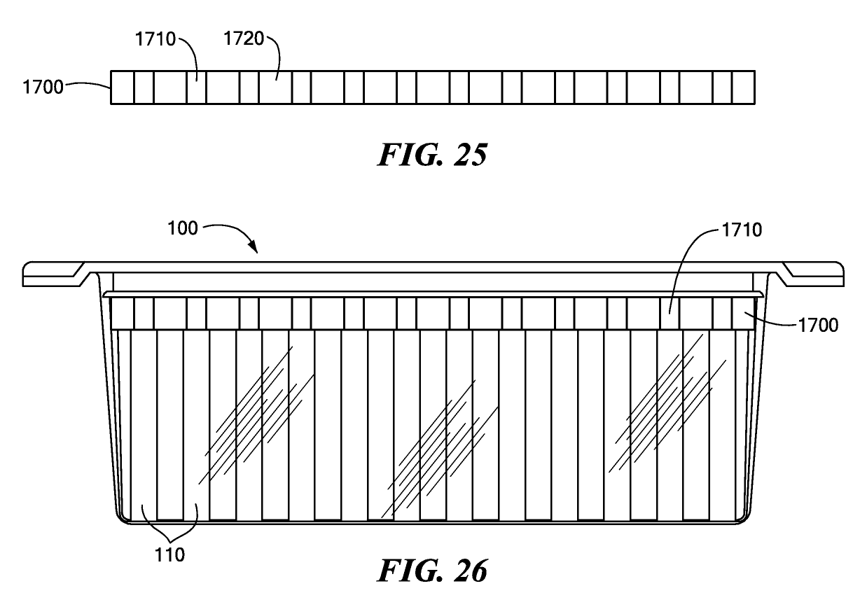

FIG. 25 shows a top view of an isoelectric focusing (IEF) membrane 1700 for use with embodiments of the invention. The IEF membrane 1700 is of the type that has immobilized ampholytes to create an immobilized pH gradient (based on the acidity constants of the immobilized buffers). When a sample containing proteins is applied to the IEF membrane 1700 and exposed to an electric field, the proteins migrate to a region of the membrane having a pH that balances the protein's charge; migration consequently ceases at that point. In a manner unlike conventional pH gradient membranes, the IEF membrane 1700 has ampholytes that are entirely or substantially grouped in zones 1710 along the length of the IEF membrane 1700. These zones correspond to the spacing of a complementary cassette. Each zone 1710 may have a single pH value, or subset of the pH gradient found in a conventional membrane. Multiple zones 1710 along the IEF membrane 1700 may together form a pH gradient separated by intervening regions 1720. The intervening regions 1720 may lack immobilized buffers. Optionally. the zones 1710 may be overlapping in pH range.

As shown in FIG. 26, the IEF membrane 1700 may be positioned atop an electrophoresis cassette 100. Application of any appropriate reagents, such as SDS for SDS-PAGE, and application of an electric field will cause migrations of proteins from the IEF membrane into the gels 110 for electrophoretic separation. Application of the membrane may be facilitated by placing the IEF membrane in a carrier, effecting electrofocusing, and then applying the carrier directly atop the cassette.

Alternately, individual samples may be held in individual capillaries (item 1500 of FIGS. 23-24). In this embodiment, a negative pressure source would not be necessary since capillary-action should be sufficient to introduce samples into the individual capillaries. A positive or negative pressure source may be required to dispense samples, or they may be wicked out of the capillaries 1500 (e.g., DNA samples to a piece of Whatman FTA® paper).

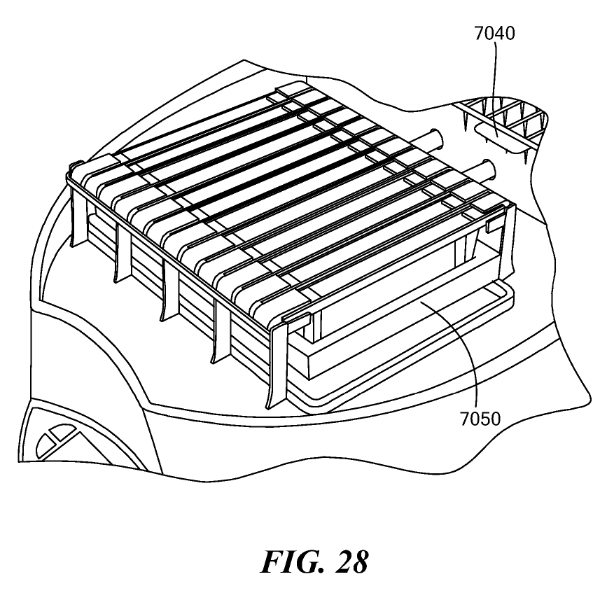

FIG. 27 shows a perspective view of an instrument 7000 for performing parallel electrophoresis using cassettes 100. The instrument 7000 accepts cassettes 100, which slide through an upper support member 7060 and into a lower support member and 7050. FIG. 28 shows a close-up view of the region of the instrument 7000 that includes the upper support member 7060 and the lower support member 7050. The upper support member may include hinged tabs 7020 to hold the cassettes 100 in place. The lower support member 7040 may include one or more electrophoresis electrodes and, optionally, detection optics (e.g., fiber optic-based fluorescence sensors). The lower support member 7050, may also accept a supply of electrophoresis buffer. A safety lid 7010 is shown here in the open position. Controls 7030 may be used to program electrophoresis time and power settings. An electrophoresis power supply and/or a thermoelectric cooler may be built into the base of the instrument 7000.

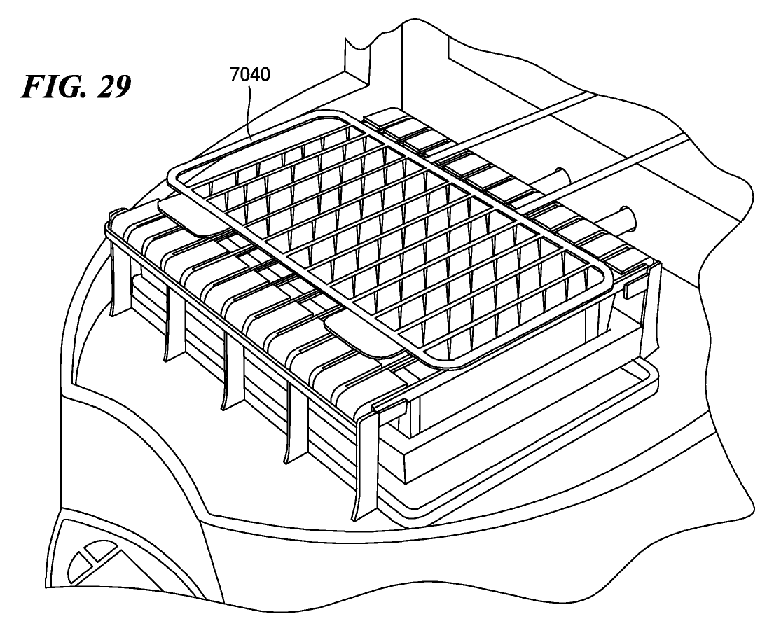

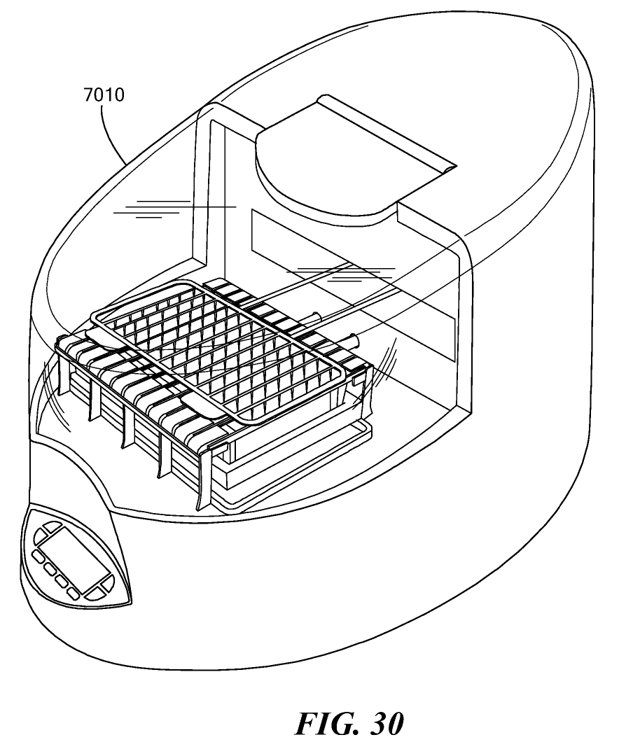

The instrument 7000 may have a pull-out upper electrode 7040. FIG. 28 shows the electrode 7040 in a retracted position and FIG. 29 shows the upper electrode 7040 in an extended position to complete the electrophoresis circuit. The electrode may be extended by pulling on its tabs, or may be automatically extended upon closing the safety lid 7010. FIG. 30 shows the instrument 7000 with the lid closed. To prevent electric shock hazard to the user, current may be switched on only when the lid is closed.

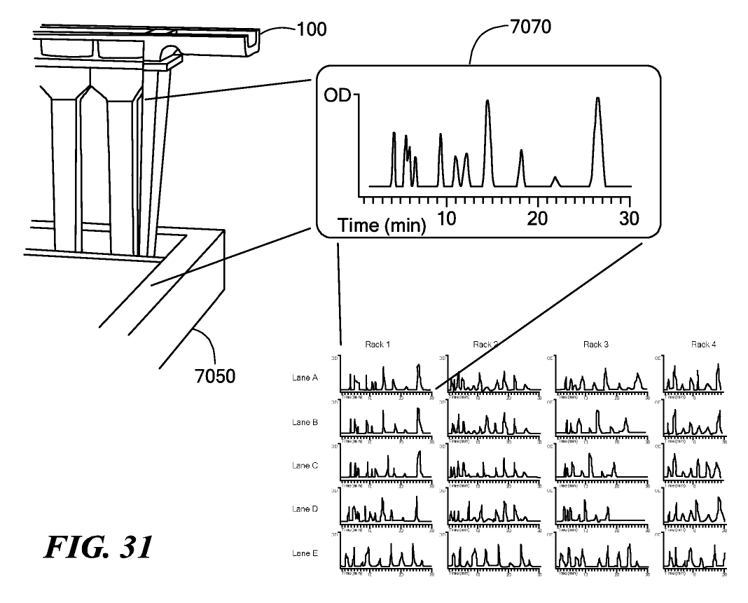

FIG. 31 shows how multiple electropherograms may be simultaneously generated in accordance with embodiments of the invention. If an instrument 7000 is equipped with optical sensors (e.g., fluorescence detection optics) positioned in the lower support member 7050, and a suitable dye is added to the sample or gel (e.g., fluorescent such as SYBR Green for nucleic acid electrophoresis), the sensors will detect analyte molecules as they travel past the sensors. Accordingly, multiple electropherograms may be generated in parallel (e.g. 96). If the gel 110 includes an adjacent membrane during electrophoresis, the membrane should be transparent, or include a slot or window so as not to distort or occlude optical measurement of the bands.

It should be recognized by one of ordinary skill in the art that the apparatus and methods described herein will be useful in a wide variety of applications in the chemical and life sciences. Molecular biology applications in the area of DNA analysis and preparation include: analysis of PCR and RT-PCR products including multiplex PCR analysis, restriction digest separation including RFLP analysis, southern blots, heteroduplex analysis using mismatch cleavage enzymes, cloning experiments and quality control of sequencing templates. Molecular biology applications in the area of RNA analysis and preparation include: northern blot analysis, analysis of RT-PCR products, expression profiling using DNA arrays, in-vitro RNA transcription assays, and preparation of cDNA libraries. Applications in the area of protein analysis and preparation include: 2-dimensional electrophoresis, western blotting, checking cell lysates for recombinant protein expression including identifying over-expressed proteins, comparing different expression patterns, purifying proteins, identify proteins of interest, monitoring protein isolation and purification processes, checking purification fractions for impurities, optimizing purification protocols. Applications involving antibodies include: monitoring impurities in antibody preparations, checking the integrity of monoclonal and polyclonal antibodies, and parallel analysis of antibodies under reducing and non-reducing conditions.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, and digital signal processors), or other related components.

In an alternative embodiment, the disclosed apparatus and methods may be implemented as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium.

The medium may be either a tangible medium (e.g., optical or analog communications lines) or a medium implemented with wireless techniques (e.g., WIFI, microwave, infrared or other transmission techniques). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software.

Although the above discussion discloses various exemplary embodiments of the invention, it should be apparent that those skilled in the art can make various modifications that will achieve some of the advantages of the invention without departing from the true scope of the invention.

What is claimed is:

1. A combined electrophoresis and blotting assembly comprising:
a frame having at least one window, a first membrane and a gel adjacent the membrane, the membrane attached to the frame so as to extend across the window.

2. An apparatus according to claim 1, wherein the first membrane is a blotting membrane.

3. An apparatus according to claim 1, further comprising a blotting membrane positioned between the first membrane and the gel.

4. An assembly according to claim 1, wherein the frame has a plurality of windows.

5. An assembly according to claim 1, wherein the membrane is attached to frame via polymeric material of the frame dissolved within pores of the membrane adjacent the frame.

6. An assembly according to claim 1, wherein the membrane is chemically tensioned across the window.

7. A combined electrophoresis and blotting assembly comprising:
a first gel, a blotting membrane layered upon the gel, wherein the blotting membrane is affixed to the gel by a second peelable gel.

8. An assembly according to claim 7, wherein the first gel is a polyacrylamide gel and the second peelable gel is an agarose gel.

9. A structure for sequential electrophoresis and electroblotting, the structure comprising:
a gel cast between two membranes, each membrane solvent-welded and chemically tensioned to a frame.

10. A structure according to claim 9, wherein at least one membrane is coated with a release agent.

11. A method for performing electrophoresis and blotting, the method comprising:
providing an electrophoresis gel and a blotting membrane adjacent the gel, the membrane defining an electrophoresis plane;
immersing the gel and membrane in an electrically insulating liquid;
applying a first electric field having at least a component oriented along the electrophoresis plane, the field being of sufficiently high voltage to cause electrophoretic mobility of charged analyte molecules in the gel;
replacing the insulating liquid with an electrically conductive liquid; and
applying a second electric field having a component normal to the electrophoretic plane the filed being of sufficiently high voltage so as to cause migration of the analyte molecules to the membrane.

12. A method according to claim 11 further comprising separating the gel from the membrane after migration of the molecules to the membrane.

13. A method according to claim 12, wherein the membrane further includes a release agent.

14. A method according to claim 11 wherein the membrane is solvent-welded to a frame, the membrane extending across at least on window.

15. A method according to claim 14 wherein the membrane is chemically tensioned.

16. A method according to claim 14 wherein the frame has a plurality of windows.

17. An electrophoresis and electroblotting instrument comprising:
a jig for holding an electrophoretic gel adjacent to a blotting membrane;
a first electrode pair oriented to apply an electrophoretic field within the gel;
a second electrode pair oriented to apply an electroblotting field across the gel and membrane;
a fluidic line having a first reservoir for holding an insulating fluid, a second reservoir for holding a conducting electrolyte fluid, conduits for transporting the insulating fluid and the conducting fluid to regions proximal to the gel and the membrane;
an automatically actuable fluid delivery assembly adapted to selectively introduce either the insulating or conducting fluid to the gel and membrane;
circuitry for sequentially actuating the introduction of insulating fluid, the first electrode pair, the introduction of conducting fluid, and the second electrode pair so as to first effectuate electrophoresis in the presence of the insulating fluid and then effectuate electroblotting in the presence of the conducting fluid.

18. An instrument according to claim 17, further comprising a cooler adapted to remove heat from one of the insulating fluid, the conductive fluid, and the gel.

19. A system for parallel gel electrophoresis comprising:
at least one cassette having a plurality of cavities for holding a plurality of electrophoretic separation matrices, each cavity having a corresponding individual sample loading port, wherein the sample loading ports are arranged with a microplate spacing.

20. A method for sample analysis and processing comprising:
(a) providing at least one cassette having a plurality of gel cavities for holding a plurality of gels, each gel cavity having a corresponding individual sample loading port; and
(b) forming a gel in the plurality of cavities;
(c) loading a plurality of samples into a plurality of corresponding loading ports; and
(d) performing electrophoresis.
wherein at least one gel is bounded by a membrane.

21. An expandable microplate-format frame comprising:
a plurality of receptacles arranged in a configuration selected from the group consisting of 8 rows of 12 receptacles, and 12 rows of 8 receptacles; and
means for increasing the distance between the rows of receptacles.

22. A system for electrophoresis comprising:
a frame having a plurality of elongate projections extending substantially parallel to a given plane, the projections defining at least one gel cavity filled by at least one corresponding gel; and
a membrane bounding the gel on at least one side, the membrane being in a plane substantially parallel to the given plane, the membrane being removably attachable to the gel.

23. A method of electrophoresis comprising:
providing a frame having a strip of electrophoretic gels bounded by and attached to a membrane;
using the gels to perform gel electrophoresis; and
removing the membrane so as to remove the electrophoretic gels attached to the membrane from the frame.

24. An electrophoresis system comprising:
at least one electrophoretic gel having a first terminus and a second terminus bounding a continuous, non-linear gel path;
a first upward-opening port for holding a liquid, the bottom of the first port bounded by the first terminus of the gel to form a first well;
a second upward-opening port for holding a liquid, the bottom of the second port bounded by the second terminus of the gel to form a second well,
wherein the nadir of the gel path is below either one of the first terminus or the second terminus.

25. A system for two dimensional electrophoresis comprising,
an elongate immobilized pH gradient member;
a complementary parallel electrophoresis cassette, the cassette having:
a plurality of longitudinally arranged gel cavities for holding a plurality of separation matrices, at least one cavity having a corresponding individual sample loading port having walls, the plurality of gel cavities and sample loading ports in lateral arrangement; and
means for transferring biomolecules held in proximity to the immobilized pH gradient member to at least one separation matrix.

26. A method for two dimensional electrophoresis comprising:
using an elongate immobilized pH gradient member to isoelectrically separate a macromolecular mixture;
transferring the elongate member to a parallel electrophoresis cassette so that different regions of the member contact separation matrices held within the cassette; and
applying an electric field to cause migration of biomolecules from the member into at least one matrix.

27. A device for performing parallel electrophoresis, the device comprising:
a support member adapted a hold a cassette, the cassette having a plurality of parallel spaced apart electrophoresis gels, the support member having a lower electrode;
an upper electrode retractably positionable against the cassette; and
a safety lid adapted to prevent a electric shock hazard condition.

28. A device according to claim 27, further comprising a plurality of optical detectors adapted to generate a plurality of electropherograms derived from samples electrophoresed in the gels.

29. A combined electrophoresis and blotting assembly comprising:
an electrophoresis gel and a blotting membrane adjacent the gel, the blotting membrane coated with a release agent so as to allow facile separation of the membrane and the gel even after use in an electrophoresis and a blotting process.
